(19) United States
(12) Patent Application Publication
(10) Pub. No.: US 2025/0042855 A1
(43) Pub. Date: Feb. 6, 2025

(54) PERIPHERALLY AND LUMINALLY-RESTRICTED INHIBITORS OF THE SEROTONIN TRANSPORTER AS TREATMENTS FOR DISORDERS OF GASTROINTESTINAL MOTILITY AND GUT-BRAIN AXIS

(71) Applicants: The Johns Hopkins University, Baltimore, MD (US); Lieber Institute, Inc., Baltimore, MD (US)

(72) Inventors: Pankaj PASRICHA, Baltimore, MD (US); James BARROW, Baltimore, MD (US); Pablo DE LEON, Baltimore, MD (US); Yifang HUANG, Baltimore, MD (US); Qian LI, Baltimore, MD (US)

(21) Appl. No.: 18/707,244

(22) PCT Filed: Dec. 1, 2022
(86) PCT No.: PCT/US2022/080746

§ 371 Date: May 3, 2024

Related U.S. Application Data
(60) Provisional application No. 63/285,176, filed on Dec. 2, 2021.

(51) Int. Cl. C07D 211/34; A61K 31/451; A61K 31/4525; C07D 405/06

(52) U.S. Cl. CPC C07D 211/34; A61K 31/451; A61K 31/4525; C07D 405/06

(57) ABSTRACT

Compounds and method for treating a disease, condition, or disorder associated with serotonin (5-HT) signaling, including gastroenterological disorders, such as colitis, irritable bowel syndrome (IBS), constipation, diarrhea, and gastroparesis, or extra-gastrointestinal disorders, such as asthma, migraine, itching, and osteoporosis. In some aspects, the compounds inhibit serotonin/5-HIT transporter (SERT).

### BACKGROUND

Approximately 95% of the bioavailable serotonin (5-hydroxytryptamine (5-HT)) is located in the enterochromaffin (EC) cells of the bowel. 5-HT is a critical messenger for gastrointestinal fluid secretion, motility, and sensation. 5-HT is produced by specialized cells lining the gut, known as enteroendocrine cells (EEC), and plays a key role in the peristaltic reflex, which is necessary for normal passage of a bolus nutrients in the gut.

The peristaltic reflex includes active contraction behind the bolus to generate a pressure head and active relaxation in front of the bolus to allow unimpeded passage. The peristaltic reflex, like all other features of gastrointestinal motility, is under the direct control of the myenteric plexus, a large group of neurons that comprise the enteric nervous system and is contained entirely within the wall of the gut. Three main neuronal components of the myenteric plexus are involved in the peristaltic reflex. The first, known as the intrinsic primary afferent neuron (IPAN), activates an inter-neuronal circuitry that goes both up and down the gastrointestinal segment, resulting in contraction and relaxation, respectively. The IPAN therefore is a key neural element in initiating this reflex. In turn, the IPAN is activated by the release of 5-HT from the lining cells in response to stimulation by factors in the gut (such as food products, other chemicals, and mechanical distention). Grider, 2003.

Apart from a positive effect on motility, 5-HT signaling in the gut has several other important effects including secretion, vasodilation and perception of pain or nausea, neuroprotection through activation of specific 5-HT receptors located on intrinsic or extrinsic nerves, epithelial and other cell types. Changes in signaling have been implicated in a variety of gastroenterological disorders, such as colitis, irritable bowel syndrome (IBS), constipation, diarrhea, and gastroparesis. In addition, gut-derived 5-HT may play a role in extra-gastrointestinal disorders, such as asthma, migraine, itching, osteoporosis, and the like. Thus, release of 5-HT from the gut and its modulation may have several therapeutic indications (Mawe and Hoffman, 2013).

An important method to increase local 5-HT levels in the gut is by affecting its fate after release. Once 5-HT is released, it is rapidly taken back again by the enteroendocrine cells that produced it in the first place, a process called “re-uptake.” This process is necessary to terminate signaling, as well as replete the stores for the next wave of peristalsis. Re-uptake is a function of a specialized molecule called the serotonin/5-HT transporter (SERT). Inhibition of SERT therefore is expected to increase extracellular concentrations of 5-HT resulting in augmentation and persistent effects on gastrointestinal motility. Indeed, SERT inhibitors (e.g., serotonin specific reuptake inhibitors or SSRIs), which are widely used to treat depression, also are effective on serotonin signaling in the GI tract, resulting in significant gastrointestinal effects in many patients (Wang et al., 2018).

Currently available SSRIs, however, are designed to penetrate the CNS and have psychotropic and other effects that are unnecessary and become dose-limiting for disorders where only the gastrointestinal tract is intended to be targeted. A better SSRI for this purpose therefore would be one that is peripherally restricted (i.e., is available systemically, but does not cross the blood-brain barrier). Elevated systemic 5-HT levels, however, may still have adverse effects because of the action of this molecule on platelets, bone and other organs. An ideal SSRI for treatment of gastrointestinal disorders therefore is one that is luminally restricted (i.e., is available neither systemically or in the CNS and is confined to the inside of the gastrointestinal tract). The latter property is still of therapeutic benefit because SERT is expressed on the lumen-facing side of the epithelial lining cells and even if the drug is not absorbed, it can still inhibit SERT, affecting local 5-HT concentrations.

### SUMMARY

In some aspects, the presently disclosed subject matter provides a compound of formula (I):

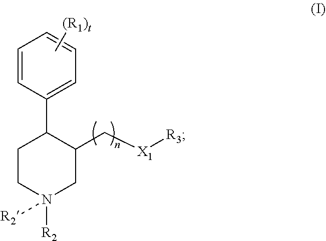

wherein:
( - - - ) indicates that the bond is present or absent;
n is an integer selected from 0, 1, 2, 3, and 4;
t is an integer selected from 0, 1, 2, and 3;
X₁ is oxygen or —CR₃R₄—, wherein R₃ and R₄ are each independently selected from H, substituted or unsubstituted straightchain or branched C₁-C₄ alkyl, halogen, substituted or unsubstituted aryl, alkoxyl, hydroxyl, carboxyl, nitro, amino, alkylamino, dialkylamino, sulfate, cyano, and mercapto;
each R₁ is independently selected from the group consisting of H, C₁-C₄ alkyl, C₁-C₂ alkoxyl, and halogen;
R₂ is H or substituted or unsubstituted straightchain or branched C₁-C₄ alkyl;
R₂′ is present or absent and, when present, is substituted or unsubstituted straightchain or branched C₁-C₄ alkyl, wherein, when present, the nitrogen atom to which it is bound has a positive charge;
R₃ is:

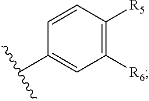

wherein:
R₅ and R₆ are each independently selected from the group consisting of H, —O—R₇, and —C(═O)—R₈, provided that at least one of R₅ or R₆ is not H, and wherein:
R₇ is selected from the group consisting of H, substituted or unsubstituted straightchain or branched C₁-C₄ alkyl, and —(CH₂)ₘ—R₉, wherein m is an integer selected from the group consisting of 0, 1, 2, 3, 4, 5, 6, 7, and 8;
R₉ is selected from the group consisting of —OR₁₀, —C(═O)—R₁₁, and —NR₁₂R₁₃, wherein:
R₁₀ is H or —(CH₂)ₚ—R₁₄, wherein p is an integer selected from the group consisting of 0, 1, 2, 3, 4, 5, 6, 7, and 8, R₁₄ is —OR₁₅ or —C(═O)—R₁₆, wherein R₁₅ and R₁₆ are each independently H or substituted or unsubstituted straightchain or branched C₁-C₄ alkyl;
R₁₁ is H or substituted or unsubstituted straightchain or branched C₁-C₄ alkyl;
R₁₂ and R₁₃ are each independently H or substituted or unsubstituted straightchain or branched C₁-C₄ alkyl;
R₈ is —OR 17 or —NR₁₈—(CH₂)_{q}—R₁₉, wherein:
q is an integer selected from 0, 1, 2, 3, 4, 5, 6, 7, and 8;
R₁₇ and R₁₈ are each independently H or substituted or unsubstituted straightchain or branched C₁-C₄ alkyl;
R₁₉ is —NR₂₀R₂₁ or —N═CR₂₂R₂₃, wherein:
R₂₀ and R₂₁ are each independently selected from the group consisting of H, substituted or unsubstituted straightchain or branched C₁-C₄ alkyl, and —C(═O)—NR₂₄R₂₅, wherein R₂₄ and R₂₅ are each independently H or substituted or unsubstituted straightchain or branched C₁-C₄ alkyl C₁-C₄ alkyl; and
R₂₂ and R₂₃ are each independently-NR₂₆R₂₇, wherein R₂₆ and R₂₇ are each independently H or substituted or unsubstituted straightchain or branched C₁-C₄ alkyl C₁-C₄ alkyl; or
wherein R₅ and R₆ together form a 5-membered heterocylic ring along with two carbons of the phenyl ring to which they are bound; and
pharmaceutically acceptable salts thereof.

In certain aspects, the compound of formula (I) is:

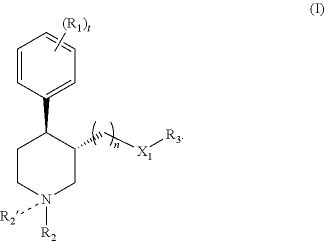

In certain aspects, the compound of formula (I) is selected from the group consisting of:

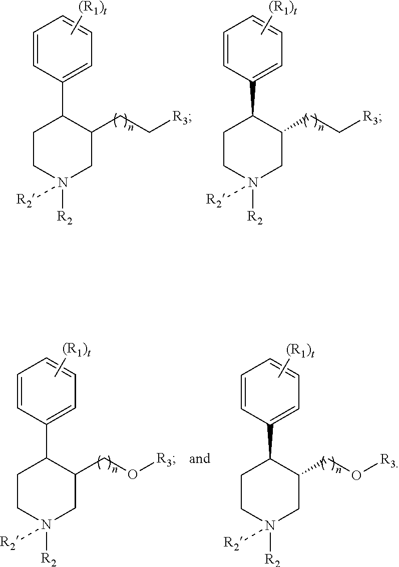

In certain aspects of the compound of formula (I), R₅ is H and R₆ is —O—R₇ or —C(═O)—R₈; or R₆ is H and R₅ is —O—R₇ or —C(═O)—R₈.

In certain aspects of the compound of formula (I):
n is 1;
X₁ is oxygen or —CR₃R₄—, wherein R₃ and R₄ are each H;
R₁ is halogen;
R₂ is H or substituted or unsubstituted straightchain or branched C₁-C₄ alkyl;
R₂′ is present or absent and, when present, is substituted or unsubstituted straightchain or branched C₁-C₄ alkyl, wherein, when present, the nitrogen atom to which it is bound has a positive charge;
R₃ is:

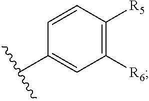

wherein:
R₆ is selected from the group consisting of H, —OH, and —C(═O)—OH;
R₅ is selected from the group consisting of H, —O—R₇, and —C(—O)—R₈;
R₇ is substituted or unsubstituted straightchain or branched C₁-C₄ alkyl or (CH₂)ₘ—R₉, wherein m is an integer selected from the group consisting of 0, 1, 2, 3, 4, 5, 6, 7, and 8;
R₉ is selected from the group consisting of —OR₁₀, —C(═O)—R₁₁, and —NR₁₂R₁₃, wherein:
R₁₀ is —(CH₂)ₚ—R₁₄, wherein p is an integer selected from the group consisting of 0, 1, 2, 3, 4, 5, 6, 7, and 8, R₁₄ is —OR₁₅ or —C(═O)—R₁₆, wherein R₁₅ and R₁₆ are each H;
R₁₁ is H;
R₁₂ and R₁₃ are each independently H or substituted or unsubstituted straightchain or branched C₁-C₄ alkyl;
R₈ is —OR₁₇ or —NR₁₈—(CH₂)_{q}—R₁₉, wherein:
q is an integer selected from 0, 1, 2, 3, 4, 5, 6, 7, and 8;
R₁₇ and R₁₈ are each H;
R₁₉ is —NR₂₀R₂₁ or —N—CR₂₂R₂₃, wherein:
R₂₀ and R₂₁ are each independently selected from the group consisting of H, substituted or unsubstituted straightchain or branched C₁-C₄ alkyl, and —C(═O)—NR₂₄R₂₅, wherein R₂₄ and R₂₅ are each H; and
R₂₂ and R₂₃ are each independently-NR₂₆R₂₇, wherein R₂₆ and R₂₇ are each H; or
wherein R₅ and R₆ together form a 5-membered heterocylic ring along with two carbons of the phenyl ring to which they are bound; and
pharmaceutically acceptable salts thereof.

In certain aspects of the compound of formula (I):
R₆ is H and R₅ is —C(═O)—R₈;
R₈ is —OR 17 or —NR₁₈—(CH₂)_{q}—R₁₉, wherein:
q is 2;
R₁₇ and R₁₈ are each H;
R₁₉ is —NR₂₀R₂₁ or —N═CR₂₂R₂₃, wherein:
R₂₀ and R₂₁ are each independently selected from the group consisting of H, substituted or unsubstituted straightchain or branched C₁-C₄ alkyl, and —C(═O)—NR₂₄R₂₅, wherein R₂₄ and R₂₅ are each H; and
R₂₂ and R₂₃ are each independently-NR₂₆R₂₇, wherein R₂₆ and R₂₇ are each H.

In certain aspects of the compound of formula (I):
R₆ is H and R₅ is —O—R₇, wherein:
R₇ is —(CH₂)ₘ—R₉, wherein m is 1, 2, or 3;
R₉ is selected from the group consisting of —OR₁₀, —C(═O)—R₁₁, and —NR₁₂R₁₃, wherein:
R₁₀ is —(CH₂)ₚ—R₁₄, wherein p is 1 or 2; R₁₄ is —OR₁₅ or —C(═O)—R₁₆, wherein R₁₅ and R₁₆ are each H;
R₁₁ is H; and
R₁₂ and R₁₃ are each independently H or substituted or unsubstituted straightchain or branched C₁-C₄ alkyl.

In certain aspects of the compound of formula (I), R₅ and R₆ together form a 1,3-dioxolane ring with two carbons of the phenyl group to which they are attached.

In particular aspects, the compound of formula (I) is selected from the group consisting of: (3S,4R)-3-(2-(benzo[d][1,3]dioxol-5-yl)ethyl)-4-(4-fluorophenyl)-1,1-dimethylpiperidin-1-ium iodide; 5-(((3S,4R)-4-(4-fluorophenyl)piperidin-3-yl)methoxy)-2-methoxybenzoic acid; 4-(((3S,4R)-4-(4-fluorophenyl)piperidin-3-yl)methoxy)-2-hydroxybenzoic acid; 3-(((3S,4R)-4-(4-fluorophenyl)piperidin-3-yl)methoxy)benzoic acid; 4-(((3S,4R)-4-(4-fluorophenyl)piperidin-3-yl)methoxy)benzoic acid; N-(2-((diaminomethylene)amino)ethyl)-4-(((3S,4R)-4-(4-fluorophenyl)piperidin-3-yl)methoxy)benzamide; 4-(((3S,4R)-4-(4-fluorophenyl)piperidin-3-yl)methoxy)-N-(2-ureidoethyl)benzamide; N-(2-aminoethyl)-4-(((3S,4R)-4-(4-fluorophenyl)piperidin-3-yl)methoxy)benzamide; N-(2-(dimethylamino)ethyl)-4-(((3S,4R)-4-(4-fluorophenyl)piperidin-3-yl)methoxy)benzamide; N-(2-(dimethylamino)ethyl)-4-(((3S,4R)-4-(4-fluorophenyl)piperidin-3-yl)methoxy)-N-methylbenzamide; 3-(4-(((3S,4R)-4-(4-fluorophenyl)piperidin-3-yl)methoxy)phenoxy)-N,N-dimethylpropan-1-amine; 2-(2-(4-(((3S,4R)-4-(4-fluorophenyl)piperidin-3-yl)methoxy)phenoxy)ethoxy)ethan-1-ol; 2-(2-(4-(((3S,4R)-4-(4-fluorophenyl)piperidin-3-yl)methoxy)phenoxy)ethoxy)acetic acid; 2-(4-(((3S,4R)-4-(4-fluorophenyl)piperidin-3-yl)methoxy)phenoxy)ethan-1-amine; 2-(4-(((3S,4R)-4-(4-fluorophenyl)piperidin-3-yl)methoxy)phenoxy)-N,N-dimethylethan-1-amine; 2-(4-(((3S,4R)-4-(4-fluorophenyl)piperidin-3-yl)methoxy)phenoxy)ethan-1-ol; and 2-(4-(((3S,4R)-4-(4-fluorophenyl)piperidin-3-yl)methoxy)phenoxy)acetic acid.

In more particular aspects, the compound of formula (I) comprises a pharmaceutically acceptable salt selected from the group consisting of:

4-(((3S,4R)-4-(4-fluorophenyl)piperidin-3-yl)methoxy)benzoic acid hydrochloride; 3-(((3S,4R)-4-(4-fluorophenyl)piperidin-3-yl)methoxy)benzoic acid hydrochloride; 4-(((3S,4R)-4-(4-fluorophenyl)piperidin-3-yl)methoxy)-2-hydroxybenzoic acid hydrochloride; 5-(((3S,4R)-4-(4-fluorophenyl)piperidin-3-yl)methoxy)-2-methoxybenzoic acid hydrochloride; N-(2-aminoethyl)-4-(((3S,4R)-4-(4-fluorophenyl)piperidin-3-yl)-methoxy)benzamide hydrochloride; N-(2-(dimethylamino)ethyl)-4-(((3S,4R)-4-(4-fluorophenyl)piperidin-3-yl)methoxy)benzamide hydrochloride; N-(2-(dimethylamino)ethyl)-4-(((3S,4R)-4-(4-fluorophenyl)piperidin-3-yl)methoxy)-N-methylbenzamide hydrochloride; N-(2-((diaminomethylene)amino)ethyl)-4-(((3S,4R)-4-(4-fluorophenyl)piperidin-3-yl)methoxy)benzamide; 4-(((3S,4R)-4-(4-fluorophenyl)piperidin-3-yl)methoxy)-N-(2-ureidoethyl)benzamide hydrochloride; 2-(4-(((3S,4R)-4-(4-fluorophenyl)piperidin-3-yl)methoxy)phenoxy)-ethan-1-amine hydrochloride; 2-(4-(((3S,4R)-4-(4-fluorophenyl)piperidin-3-yl)methoxy)phenoxy)-N,N-dimethylethan-1-amine hydrochloride; 3-(4-(((3S,4R)-4-(4-fluorophenyl)piperidin-3-yl)methoxy)phenoxy)-N,N-dimethylpropan-1-amine hydrochloride; 2-(4-(((3S,4R)-4-(4-fluorophenyl)piperidin-3-yl)methoxy)phenoxy)ethan-1-ol hydrochloride; 2-(2-(4-(((3S,4R)-4-(4-fluorophenyl)piperidin-3-yl)methoxy)phenoxy)ethoxy)ethan-1-ol hydrochloride; 2-(4-(((3S,4R)-4-(4-fluorophenyl)piperidin-3-yl)methoxy)phenoxy)acetic acid hydrochloride; 2-(2-(4-(((3S,4R)-4-(4-fluorophenyl)piperidin-3-yl)methoxy)phenoxy)-ethoxy)acetic acid hydrochloride; and (3S,4R)-3-(2-(benzo[d][1,3]dioxol-5-yl)ethyl)-4-(4-fluorophenyl)-1,1-dimethylpiperidin-1-ium iodide.

In other aspects, the presently disclosed subject matter provides a pharmaceutical formulation comprising a compound of formula (I).

In yet other aspects, the presently disclosed subject matter provides a method for treating a disease, condition, or disorder associated with serotonin (5-HT) signaling, the method comprising administering a therapeutically effective compound of formula (I), and pharmaceutically acceptable salts thereof, to a subject in need of treatment thereof.

In certain aspects, the method comprises inhibiting serotonin/5-HT transporter (SERT).

In particular aspects, inhibiting SERT increases an extracellular concentration of 5-HT.

In certain aspects, the disease, condition, or disorder associated with 5-HT signaling comprises a gastroenterological disorder. In particular aspects, the gastroenterological disorder is selected from colitis, irritable bowel syndrome (IBS), constipation, diarrhea, and gastroparesis.

In certain aspects, the disease, condition, or disorder associated with 5-HT signaling comprises an extra-gastrointestinal disorder. In particular aspects, the extra-gastrointestinal disorder is selected from asthma, migraine, itching, osteoporosis, anxiety, depression and impaired cognition.

Certain aspects of the presently disclosed subject matter having been stated hereinabove, which are addressed in whole or in part by the presently disclosed subject matter, other aspects will become evident as the description proceeds when taken in connection with the accompanying Examples as best described herein below.

### BRIEF DESCRIPTION OF THE FIGURE

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying FIGURE, which is not necessarily drawn to scale, and wherein:

FIG. 1 demonstrates that chronic treatment with N-(2-((diaminomethylene)amino)ethyl)-4-(((3S,4R)-4-(4-fluorophenyl)piperidin-3-yl)methoxy)benzamide increases liquid gastric emptying. Data are presented as Mean±SEM (n=6-7 mice). *: Significant difference from H₂O treated group in the same model P<0.05.

### DETAILED DESCRIPTION

The presently disclosed subject matter now will be described more fully hereinafter. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

### I. Peripherally and Luminally-Restricted Inhibitors of the Serotonin Transporter as Treatments for Disorders of Gastrointestinal Motility and Gut-Brain Axis

Serotonin (5-hydroxytryptamine (5-HT)) is a critical messenger for gastrointestinal fluid secretion, motility, and sensation. As used herein, “gastrointestinal (GI) motility” generally refers to the movement of food from the mouth through the pharynx (throat), esophagus, stomach, small and large intestines and out of the body. Approximately 95% of the bioavailable 5-HT is located in the enterochromaffin (EC) cells of the bowel. To ensure efficient intercellular signaling of 5-HT, the reuptake and termination of serotonin from the effective site is necessary. In the intestinal mucosa, 5-HT may be removed through the serotonin transporter (SERT) that is expressed throughout the epithelial cells that line the luminal surface of the gut. By inhibition of SERT in the GI system with luminally and/or peripherally-restricted SERT inhibitors, the action on 5-HT on GI motility can be enhanced which may be significant in constipation-predominant forms of IBS. 5-HT also can modulate signaling to the brain via the vagus and other nerves to influence diverse sensations as well as mood, affect and cognitive functions (Engevik et al., 2021).

SERT inhibitors (e.g., SSRIs) are currently being used to treat depression, but they also are effective on serotonin signaling in the GI tract, resulting in diarrhea in 20-25% of patients. Serotonin signaling, however, also may be involved in other cell types found in the body, e.g., platelets and bone. Thus, an ideal drug for constipation would be one that is not absorbed (i.e., luminally and peripherally restricted), sparing the body of systemic and CNS effects, while targeting SERT locally.

SERT inhibitors that exhibit reduced brain penetration have been identified as anti-platelet agents (Rehavi and Gurwitz, WO 2007148341). Significant losses in potency are observed by making the quaternary salts in this instance. The presently disclosed subject matter provides more benign interventions and a more potent starting point to maximize efficacy.

More particularly, the presently disclosed subject matter provides novel compounds, which are potent inhibitors of SERT, but are largely restricted to the gastrointestinal lumen because of poor bioavailability. Such compounds can be used, for example, to treat gastroenterological disorders, such as gastroparesis.

Gastroparesis is a prototype disorder for decreased gastrointestinal motility, characterized by slow gastric emptying in the absence of mechanical obstruction and accompanied by nausea, vomiting, fullness, satiety and in severe cases, malnutrition and weight loss. The pathogenesis of gastroparesis is not fully understood, but a recent study by Wei et al., 2021, has provided considerable insight. In this study, the investigators examined gastric emptying in mice after inducing the loss of the TPH gene, which codes for tryptophan hydrolase, an enzyme that catalyzes the rate limiting step in the synthesis of 5-HT. When TPH is deleted, mice have severe delay in gastric emptying, which can be reversed by gastric administration of 5-HT. In addition, the study also found that 5-HT concentrations are significantly reduced in the stomach of patients with gastroparesis. Taken together, this study highlights the importance of 5-HT in regulating motility and also indicates that augmentation of 5-HT is potentially a viable and valuable approach to the treatment of gastroparesis. To that end, the presently disclosed compounds, in come embodiments, enhance gastric emptying and thus exhibit the potential for treating gastroenterological disorders, such as gastroparesis.

### A. Compounds of Formula (I)

More particularly, in some embodiments, the presently disclosed subject matter provides a compound of formula (I):

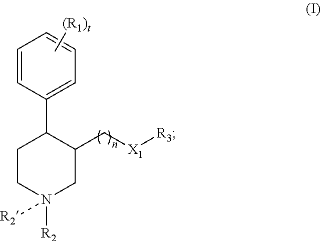

wherein:
( - - - ) indicates that the bond is present or absent;
n is an integer selected from 0, 1, 2, 3, and 4;
t is an integer selected from 0, 1, 2, and 3;
X₁ is oxygen or —CR₃R₄—, wherein R₃ and R₄ are each independently selected from H, substituted or unsubstituted straightchain or branched C₁-C₄ alkyl, halogen, substituted or unsubstituted aryl, alkoxyl, hydroxyl, carboxyl, nitro, amino, alkylamino, dialkylamino, sulfate, cyano, and mercapto;
each R₁ is independently selected from the group consisting of H, C₁-C₄ alkyl, C₁-C₂ alkoxyl, and halogen;
R₂ is H or substituted or unsubstituted straightchain or branched C₁-C₄ alkyl;
R₂′ is present or absent and, when present, is substituted or unsubstituted straightchain or branched C₁-C₄ alkyl, wherein, when present, the nitrogen atom to which it is bound has a positive charge;
R₃ is:

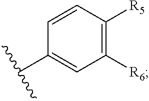

wherein:
R₅ and R₆ are each independently selected from the group consisting of H, —O—R₇, and —C(═O)—R₈, provided that at least one of R₅ or R₆ is not H, and wherein:
R₇ is selected from the group consisting of H, substituted or unsubstituted straightchain or branched C₁-C₄ alkyl, and —(CH₂)ₘ—R₉, wherein m is an integer selected from the group consisting of 0, 1, 2, 3, 4, 5, 6, 7, and 8;
R₉ is selected from the group consisting of —OR₁₀, —C(═O)—R₁₁, and —NR₁₂R₁₃, wherein:
R₁₀ is H or —(CH₂)ₚ—R₁₄, wherein p is an integer selected from the group consisting of 0, 1, 2, 3, 4, 5, 6, 7, and 8, R₁₄ is —OR₁₅ or —C(═O)—R₁₆, wherein R₁₅ and R₁₆ are each independently H or substituted or unsubstituted straightchain or branched C₁-C₄ alkyl;
R₁₁ is H or substituted or unsubstituted straightchain or branched C₁-C₄ alkyl;
R₁₂ and R₁₃ are each independently H or substituted or unsubstituted straightchain or branched C₁-C₄ alkyl;
R₈ is —OR₁₇ or —NR₁₈—(CH₂)_{q}—R₁₉, wherein:
q is an integer selected from 0, 1, 2, 3, 4, 5, 6, 7, and 8;
R₁₇ and R₁₈ are each independently H or substituted or unsubstituted straightchain or branched C₁-C₄ alkyl;
R₁₉ is —NR₂₀R₂₁ or —N═CR₂₂R₂₃, wherein:
R₂₀ and R₂₁ are each independently selected from the group consisting of H, substituted or unsubstituted straightchain or branched C₁-C₄ alkyl, and —C(═O)—NR₂₄R₂₅, wherein R₂₄ and R₂₅ are each independently H or substituted or unsubstituted straightchain or branched C₁-C₄ alkyl C₁-C₄ alkyl; and
R₂₂ and R₂₃ are each independently-NR₂₆R₂₇, wherein R₂₆ and R₂₇ are each independently H or substituted or unsubstituted straightchain or branched C₁-C₄ alkyl C₁-C₄ alkyl; or
wherein R₅ and R₆ together form a 5-membered heterocylic ring along with two carbons of the phenyl ring to which they are bound; and
pharmaceutically acceptable salts thereof.

In certain embodiments, the compound of formula (I) is:

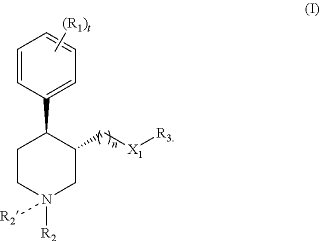

In certain embodiments, the compound of formula (I) is selected from the group consisting of:

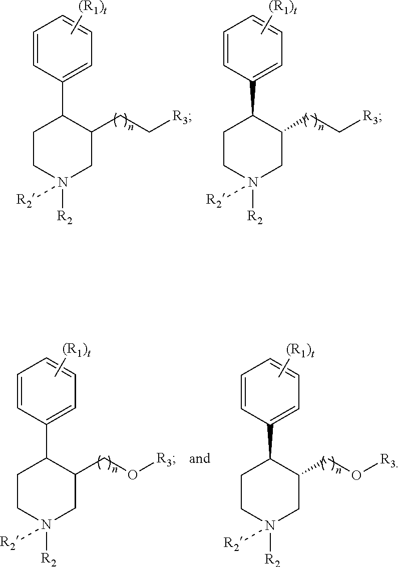

In certain embodiments of the compound of formula (I), R₅ is H and R₆ is —O—R₇ or —C(═O)—R₈; or R₆ is H and R₅ is —O—R₇ or —C(═O)—R₈.

In certain embodiments of the compound of formula (I):
n is 1;
X₁ is oxygen or —CR₃R₄—, wherein R₃ and R₄ are each H;
R₁ is halogen;
R₂ is H or substituted or unsubstituted straightchain or branched C₁-C₄ alkyl;
R₂′ is present or absent and, when present, is substituted or unsubstituted straightchain or branched C₁-C₄ alkyl, wherein, when present, the nitrogen atom to which it is bound has a positive charge;
R₃ is:

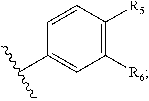

wherein:
R₆ is selected from the group consisting of H, —OH, and —C(═O)—OH;
R₅ is selected from the group consisting of H, —O—R₇, and —C(═O)—R₈;
R₇ is substituted or unsubstituted straightchain or branched C₁-C₄ alkyl or —(CH₂)ₘ—R₉, wherein m is an integer selected from the group consisting of 0, 1, 2, 3, 4, 5, 6, 7, and 8;
R₉ is selected from the group consisting of —OR₁₀, —C(═O)—R₁₁, and —NR₁₂R₁₃, wherein:
R₁₀ is —(CH₂)ₚ—R₁₄, wherein p is an integer selected from the group consisting of 0, 1, 2, 3, 4, 5, 6, 7, and 8, R₁₄ is —OR₁₅ or —C(═O)—R₁₆, wherein R₁₅ and R₁₆ are each H;
R₁₁ is H;
R₁₂ and R₁₃ are each independently H or substituted or unsubstituted straightchain or branched C₁-C₄ alkyl;
R₈ is —OR₁₇ or —NR₁₈—(CH₂)_{q}—R₁₉, wherein:
q is an integer selected from 0, 1, 2, 3, 4, 5, 6, 7, and 8;
R₁₇ and R₁₈ are each H;
R₁₉ is —NR₂₀R₂₁ or —N═CR₂₂R₂₃, wherein:
R₂₀ and R₂₁ are each independently selected from the group consisting of H, substituted or unsubstituted straightchain or branched C₁-C₄ alkyl, and —C(═O)—NR₂₄R₂₅, wherein R₂₄ and R₂₅ are each H; and
R₂₂ and R₂₃ are each independently-NR₂₆R₂₇, wherein R₂₆ and R₂₇ are each H; or
wherein R₅ and R₆ together form a 5-membered heterocylic ring along with two carbons of the phenyl ring to which they are bound; and
pharmaceutically acceptable salts thereof.

In certain embodiments of the compound of formula (I):
R₆ is H and R₅ is —C(═O)—R₈;
R& is —OR₁₇ or —NR₁₈—(CH₂)_{q}—R₁₉, wherein:
q is 2;
R₁₇ and R₁₈ are each H;
R₁₉ is —NR₂₀R₂₁ or —N═CR₂₂R₂₃, wherein:
R₂₀ and R₂₁ are each independently selected from the group consisting of H, substituted or unsubstituted straightchain or branched C₁-C₄ alkyl, and —C(═O)—NR₂₄R₂₅, wherein R₂₄ and R₂₅ are each H; and
R₂₂ and R₂₃ are each independently-NR₂₆R₂₇, wherein R₂₆ and R₂₇ are each H.

In certain embodiments of the compound of formula (I):
R₆ is H and R₅ is —O—R₇, wherein:
R₇ is —(CH₂)ₘ—R₉, wherein m is 1, 2, or 3;
R₉ is selected from the group consisting of —OR₁₀, —C(═O)—R₁₁, and —NR₁₂R₁₃, wherein:
R₁₀ is —(CH₂)ₚ—R₁₄, wherein p is 1 or 2; R₁₄ is —OR₁₅ or —C(═O)—R₁₆, wherein R₁₅ and R₁₆ are each H;
R₁₁ is H; and
R₁₂ and R₁₃ are each independently H or substituted or unsubstituted straightchain or branched C₁-C₄ alkyl.

In certain embodiments of the compound of formula (I), R₅ and R₆ together form a 1,3-dioxolane ring with two carbons of the phenyl group to which they are attached.

In particular embodiments, the compound of formula (I) is selected from the group consisting of: (3S,4R)-3-(2-(benzo[d][1,3]dioxol-5-yl)ethyl)-4-(4-fluorophenyl)-1,1-dimethylpiperidin-1-ium iodide; 5-(((3S,4R)-4-(4-fluorophenyl)piperidin-3-yl)methoxy)-2-methoxybenzoic acid; 4-(((3S,4R)-4-(4-fluorophenyl)piperidin-3-yl)methoxy)-2-hydroxybenzoic acid; 3-(((3S,4R)-4-(4-fluorophenyl)piperidin-3-yl)methoxy)benzoic acid; 4-(((3S,4R)-4-(4-fluorophenyl)piperidin-3-yl)methoxy)benzoic acid; N-(2-((diaminomethylene)amino)ethyl)-4-(((3S,4R)-4-(4-fluorophenyl)piperidin-3-yl)methoxy)benzamide; 4-(((3S,4R)-4-(4-fluorophenyl)piperidin-3-yl)methoxy)-N-(2-ureidoethyl)benzamide; N-(2-aminoethyl)-4-(((3S,4R)-4-(4-fluorophenyl)piperidin-3-yl)methoxy)benzamide; N-(2-(dimethylamino)ethyl)-4-(((3S,4R)-4-(4-fluorophenyl)piperidin-3-yl)methoxy)benzamide; N-(2-(dimethylamino)ethyl)-4-(((3S,4R)-4-(4-fluorophenyl)piperidin-3-yl)methoxy)-N-methylbenzamide; 3-(4-(((3S,4R)-4-(4-fluorophenyl)piperidin-3-yl)methoxy)phenoxy)-N,N-dimethylpropan-1-amine; 2-(2-(4-(((3S,4R)-4-(4-fluorophenyl)piperidin-3-yl)methoxy)phenoxy)ethoxy)ethan-1-ol; 2-(2-(4-(((3S,4R)-4-(4-fluorophenyl)piperidin-3-yl)methoxy)phenoxy)ethoxy)acetic acid; 2-(4-(((3S,4R)-4-(4-fluorophenyl)piperidin-3-yl)methoxy)phenoxy)ethan-1-amine; 2-(4-(((3S,4R)-4-(4-fluorophenyl)piperidin-3-yl)methoxy)phenoxy)-N,N-dimethylethan-1-amine; 2-(4-(((3S,4R)-4-(4-fluorophenyl)piperidin-3-yl)methoxy)phenoxy)ethan-1-ol; and 2-(4-(((3S,4R)-4-(4-fluorophenyl)piperidin-3-yl)methoxy)phenoxy)acetic acid.

In more particular embodiments, the compound of formula (I) comprises a pharmaceutically acceptable salt selected from the group consisting of: 4-(((3S,4R)-4-(4-fluorophenyl)piperidin-3-yl)methoxy)benzoic acid hydrochloride; 3-(((3S,4R)-4-(4-fluorophenyl)piperidin-3-yl)methoxy)benzoic acid hydrochloride; 4-(((3S,4R)-4-(4-fluorophenyl)piperidin-3-yl)methoxy)-2-hydroxybenzoic acid hydrochloride; 5-(((3S,4R)-4-(4-fluorophenyl)piperidin-3-yl)methoxy)-2-methoxybenzoic acid hydrochloride; N-(2-aminoethyl)-4-(((3S,4R)-4-(4-fluorophenyl)piperidin-3-yl)-methoxy)benzamide hydrochloride; N-(2-(dimethylamino)ethyl)-4-(((3S,4R)-4-(4-fluorophenyl)piperidin-3-yl)methoxy)benzamide hydrochloride; N-(2-(dimethylamino)ethyl)-4-(((3S,4R)-4-(4-fluorophenyl)piperidin-3-yl)methoxy)-N-methylbenzamide hydrochloride; N-(2-((diaminomethylene)amino)ethyl)-4-(((3S,4R)-4-(4-fluorophenyl)piperidin-3-yl)methoxy)benzamide; 4-(((3S,4R)-4-(4-fluorophenyl)piperidin-3-yl)methoxy)-N-(2-ureidoethyl)benzamide hydrochloride; 2-(4-(((3S,4R)-4-(4-fluorophenyl)piperidin-3-yl)methoxy)phenoxy)-ethan-1-amine hydrochloride; 2-(4-(((3S,4R)-4-(4-fluorophenyl)piperidin-3-yl)methoxy)phenoxy)-N,N-dimethylethan-1-amine hydrochloride; 3-(4-(((3S,4R)-4-(4-fluorophenyl)piperidin-3-yl)methoxy)phenoxy)-N,N-dimethylpropan-1-amine hydrochloride; 2-(4-(((3S,4R)-4-(4-fluorophenyl)piperidin-3-yl)methoxy)phenoxy)ethan-1-ol hydrochloride; 2-(2-(4-(((3S,4R)-4-(4-fluorophenyl)piperidin-3-yl)methoxy)phenoxy)ethoxy)ethan-1-ol hydrochloride; 2-(4-(((3S,4R)-4-(4-fluorophenyl)piperidin-3-yl)methoxy)phenoxy)acetic acid hydrochloride; 2-(2-(4-(((3S,4R)-4-(4-fluorophenyl)piperidin-3-yl)methoxy)phenoxy)-ethoxy)acetic acid hydrochloride; and (3S,4R)-3-(2-(benzo[d][1,3]dioxol-5-yl)ethyl)-4-(4-fluorophenyl)-1,1-dimethylpiperidin-1-ium iodide.

In particular embodiments, the compound of formula (I) is selected from the group consisting of:

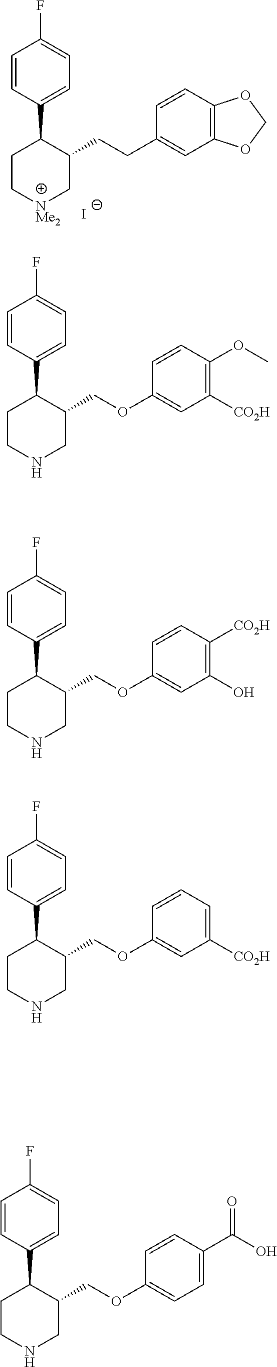

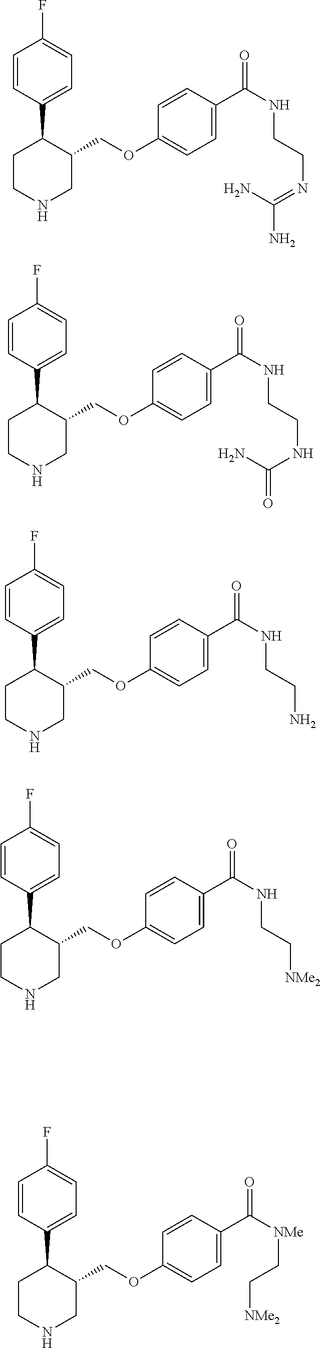 [SMILES: NC(N)=NCCNC(=O)c1ccc(OC[C@@H]2CNCC[C@H]2c2ccc(F)cc2)cc1 ; NC(=O)NCCNC(=O)c1ccc(OC[C@@H]2CNCC[C@H]2c2ccc(F)cc2)cc1 ; NCCNC(=O)c1ccc(OC[C@@H]2CNCC[C@H]2c2ccc(F)cc2)cc1 ; CN(C)CCNC(=O)c1ccc(OC[C@@H]2CNCC[C@H]2c2ccc(F)cc2)cc1 ; CN(C)CCN(C)C(=O)c1ccc(OC[C@@H]2CNCC[C@H]2c2ccc(F)cc2)cc1]

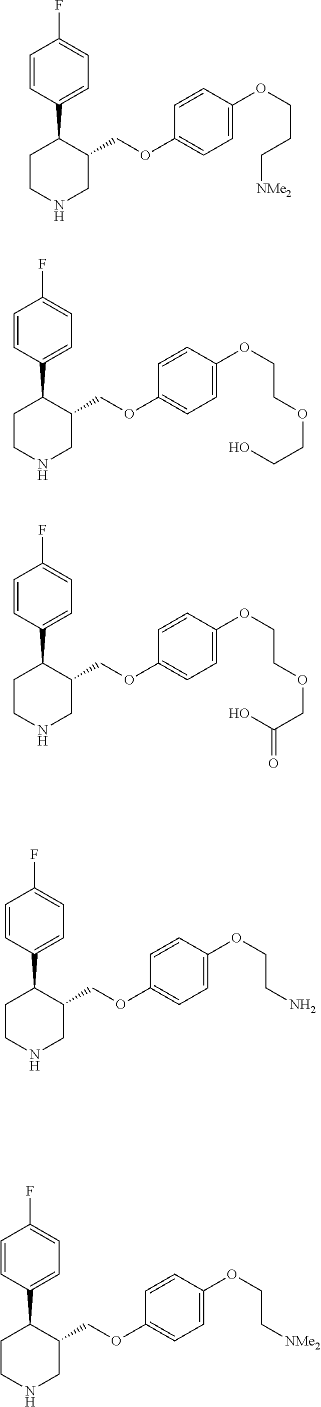 [SMILES: CN(C)CCCOc1ccc(OC[C@@H]2CNCC[C@H]2c2ccc(F)cc2)cc1 ; OCCOCCOc1ccc(OC[C@@H]2CNCC[C@H]2c2ccc(F)cc2)cc1 ; O=C(O)COCCOc1ccc(OC[C@@H]2CNCC[C@H]2c2ccc(F)cc2)cc1 ; NCCOc1ccc(OC[C@@H]2CNCC[C@H]2c2ccc(F)cc2)cc1 ; CN(C)CCOc1ccc(OC[C@@H]2CNCC[C@H]2c2ccc(F)cc2)cc1]

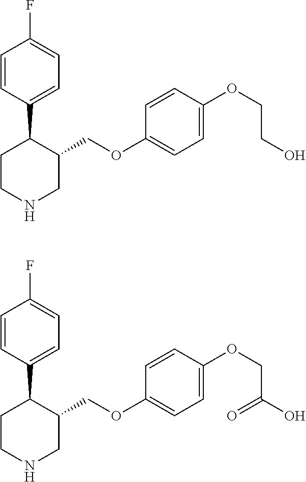 [SMILES: OCCOc1ccc(OC[C@@H]2CNCC[C@H]2c2ccc(F)cc2)cc1 ; O=C(O)COc1ccc(OC[C@@H]2CNCC[C@H]2c2ccc(F)cc2)cc1]

In other embodiments, the presently disclosed subject matter provides a pharmaceutical formulation comprising a compound of formula (I).

B. Methods for Treating a Disease, Condition, or Disorder Associated with Serotonin Signaling

In yet other embodiments, the presently disclosed subject matter provides a method for treating a disease, condition, or disorder associated with serotonin (5-HT) signaling, the method comprising administering a therapeutically effective compound of formula (I), and pharmaceutically acceptable salts thereof, to a subject in need of treatment thereof.

In certain embodiments, the method comprises inhibiting serotonin/5-HT transporter (SERT).

In particular embodiments, inhibiting SERT increases an extracellular concentration of 5-HT.

In certain embodiments, the disease, condition, or disorder associated with 5-HT signaling comprises a gastroenterological disorder. In particular embodiments, the gastroenterological disorder is selected from colitis, irritable bowel syndrome (IBS), constipation, diarrhea, and gastroparesis.

In certain embodiments, the disease, condition, or disorder associated with 5-HT signaling comprises an extra-gastrointestinal disorder. In particular embodiments, the extra-gastrointestinal disorder is selected from asthma, migraine, itching, osteoporosis, anxiety, depression and impaired cognition.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the term “inhibit,” and grammatical derivations thereof, refers to the ability of a presently disclosed compound, e.g., a presently disclosed compound of formula (I), to block, partially block, interfere, decrease, or reduce activity of a target. Thus, one of ordinary skill in the art would appreciate that the term “inhibit” encompasses a complete and/or partial decrease in the activity of a target, e.g., a decrease by at least 10%, in some embodiments, a decrease by at least 20%, 30%, 50%, 75%, 95%, 98%, and up to and including 100%.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound of Formula (I) and at least one analgesic; and, optionally, one or more analgesic agents. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds of Formula (I) described herein can be administered alone or in combination with adjuvants that enhance stability of the compounds of Formula (I), alone or in combination with one or more analgesic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound of Formula (I) and at least one additional therapeutic agent can be varied so long as the beneficial effects of the combination of these agents are achieved. Accordingly, the phrase “in combination with” refers to the administration of a compound of Formula (I) and at least one additional therapeutic agent either simultaneously, sequentially, or a combination thereof. Therefore, a subject administered a combination of a compound of Formula (I) and at least one additional therapeutic agent can receive compound of Formula (I) and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound of Formula (I) and at least one additional therapeutic agent are administered simultaneously, they can be administered to the subject as separate pharmaceutical compositions, each comprising either a compound of Formula (I) or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound of Formula (I) and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### C. Pharmaceutical Compositions and Administration

In another aspect, the present disclosure provides a pharmaceutical composition including one compound of Formula (I) alone or in combination with one or more additional therapeutic agents in admixture with a pharmaceutically acceptable excipient.

One of ordinary skill in the art will recognize that the pharmaceutical compositions include the pharmaceutically acceptable salts of the compounds described above. Pharmaceutically acceptable salts are generally well known to those of ordinary skill in the art and include salts of active compounds which are prepared with relatively nontoxic acids or bases, depending on the particular substituent moieties found on the compounds described herein.

When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another.

Examples of pharmaceutically acceptable base addition salts include sodium, potassium, calcium, zinc, magnesium, ammonium, piperidine, piperazine, organic amino, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another.

Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like.

Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19).

Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20th ed.) Lippincott, Williams & Wilkins (2000).

Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20th ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of ordinary skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### II. Chemical Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of Formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O) NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a (n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

C₁-C₄ alkyl includes C₁, C₂, C₃, and C₄ alkyl, including methyl, ethyl, propyl, isopropyl, butyl, isobutyl, sec-butyl, and t-butyl.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, carboxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur(S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; CH═CH—CH₂—; —CH₂CH₂CH₂CH₂—, —CH₂CH═CHCH₂—, —CH₂CsCCH₂—, —CH₂CH₂CH(CH₂CH₂CH₃) CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O) OR′-represents both-C(O) OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy) propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

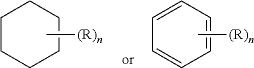

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

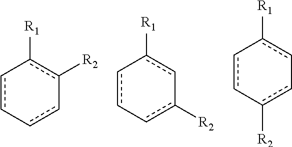

and the like.

A dashed line, e.g., [symbol] , representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, —O, ═NR′, ═N—OR′, —NR′R″, —SR′, -halogen, —SiR′R″R″ “, —OC(O) R′, —C(O) R′, —CO₂R′, —C(O)NR′R”, —OC(O)NR′R″, —NR″C(O)R′, —NR′—C(O)NR″R″, —NR″C(O)OR′, —NR—C(NR′R″)═NR′ “, —S(O)R′, —S(O)₂R′, —S(O)₂NR′R”, —NRSO₂R′, —CN, CF₃, fluorinated C₁₋₄ alkyl, and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R″″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of ordinary skill in the art will understand that the term “alkyl” is meant to include groups including carbon atoms bound to groups other than hydrogen groups, such as haloalkyl (e.g., —CF₃ and —CH₂CF₃) and acyl (e.g., —C(O) CH₃, —C(O) CF₃, —C(O) CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R″ “, —OC(O)R′, —C(O) R′, —CO₂R′, —C(O)NR′R”, —OC(O)NR′R″, —NR″C(O)R′, —NR′—C(O)NR″R″, —NR″C(O)OR′, —NR—C(NR′R″R″)═NR″ “, —NR—C(NR′R”)═NR″—S(O)R′, —S(O)₂R′, —S(O)₂NR′R″, —NRSO₂R′, —CN and —NO₂, —R′, —N₃, —CH(Ph)₂, fluoro (C₁₋₄)alkoxo, and fluoro (C₁₋₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently-NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently-CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O) NR′, esters, —RC(═O) OR′, ketones, —RC(═O) R′, and aldehydes, —RC(═O) H. The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described and include substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl. “Aryloxycarbonyl” refers to an aryl-O—C(═O)— group. Exemplary aryloxycarbonyl groups include phenoxy- and naphthoxy-carbonyl. “Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic groups. For example, the terms “acylamino” and “alkylamino” refer to specific N-substituted organic groups with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O) H.

The term “carboxyl” refers to the —COOH group. Such groups also are referred to herein as a “carboxylic acid” moiety.

The term “cyano” refers to the —C≡N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group-S(O₂)R′.

The term “sulfoxide” refers to a compound having a sulfinyl group-S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or(S)— or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example deuterium (²H), tritium (³H), iodine-125 (¹²⁵I) or carbon-14 (¹⁴C). All isotopic variations of the compounds of the present disclosure, whether radioactive or not, are encompassed within the scope of the present disclosure.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P.G.M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium (O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react. Typical blocking/protecting groups include, but are not limited to the following moieties:

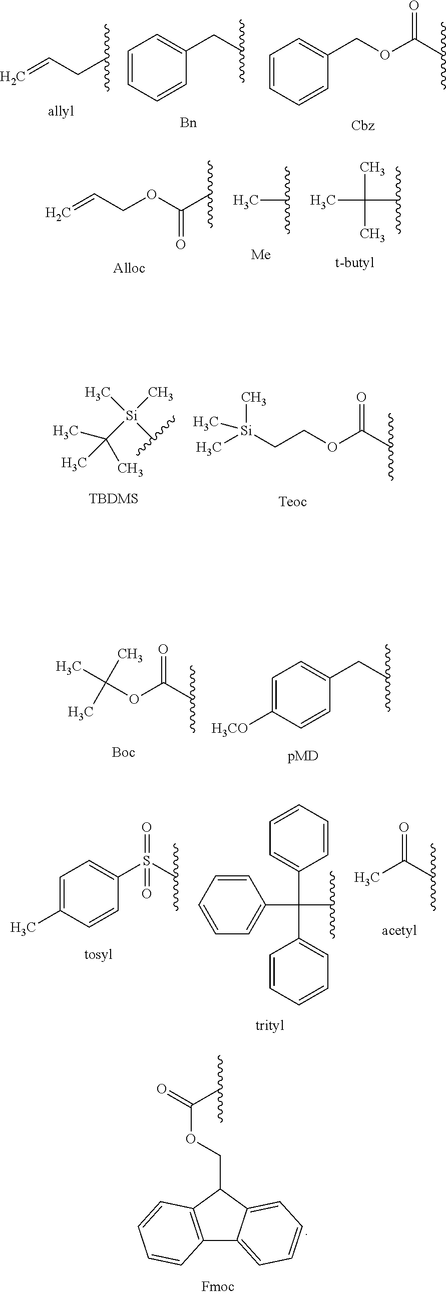

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Examples 1-17

### Representative Compounds

The following representative compounds can be prepared and evaluated according to procedures provided in the following examples:

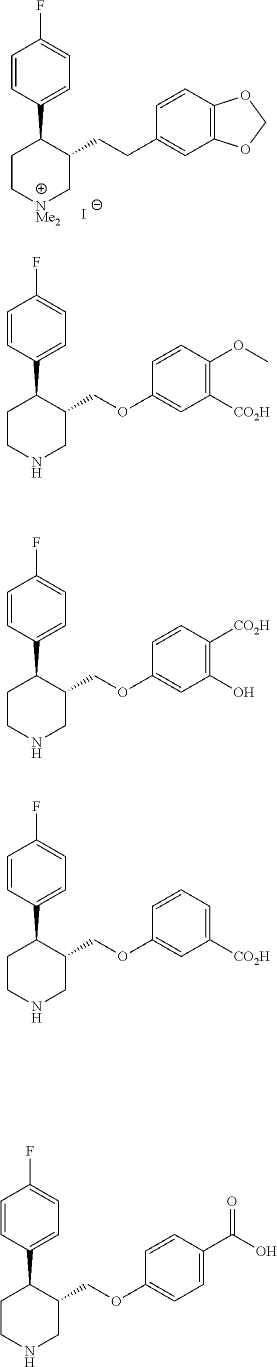

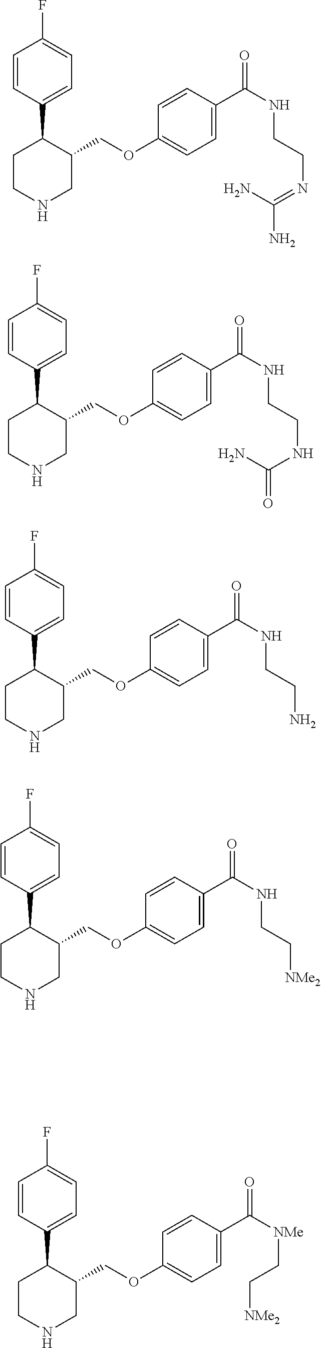 [SMILES: NC(N)=NCCNC(=O)c1ccc(OC[C@@H]2CNCC[C@H]2c2ccc(F)cc2)cc1 ; NC(=O)NCCNC(=O)c1ccc(OC[C@@H]2CNCC[C@H]2c2ccc(F)cc2)cc1 ; NCCNC(=O)c1ccc(OC[C@@H]2CNCC[C@H]2c2ccc(F)cc2)cc1 ; CN(C)CCNC(=O)c1ccc(OC[C@@H]2CNCC[C@H]2c2ccc(F)cc2)cc1 ; CN(C)CCN(C)C(=O)c1ccc(OC[C@@H]2CNCC[C@H]2c2ccc(F)cc2)cc1]

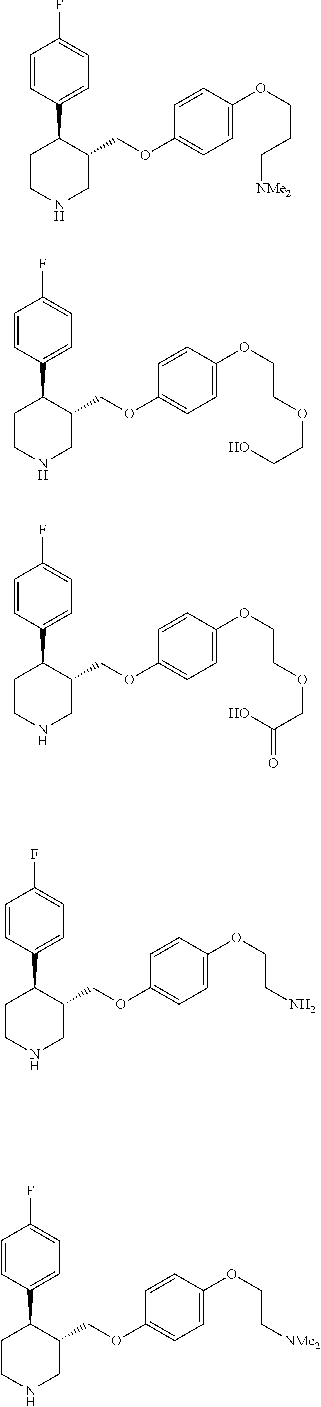 [SMILES: CN(C)CCCOc1ccc(OC[C@@H]2CNCC[C@H]2c2ccc(F)cc2)cc1 ; OCCOCCOc1ccc(OC[C@@H]2CNCC[C@H]2c2ccc(F)cc2)cc1 ; O=C(O)COCCOc1ccc(OC[C@@H]2CNCC[C@H]2c2ccc(F)cc2)cc1 ; NCCOc1ccc(OC[C@@H]2CNCC[C@H]2c2ccc(F)cc2)cc1 ; CN(C)CCOc1ccc(OC[C@@H]2CNCC[C@H]2c2ccc(F)cc2)cc1]

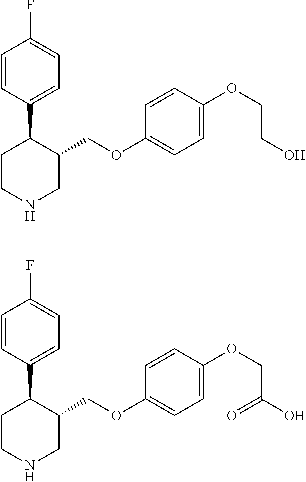 [SMILES: OCCOc1ccc(OC[C@@H]2CNCC[C@H]2c2ccc(F)cc2)cc1 ; O=C(O)COc1ccc(OC[C@@H]2CNCC[C@H]2c2ccc(F)cc2)cc1]

### Synthetic Procedures

Exemplary compounds were prepared via several general synthetic routes set forth in the examples below. Any of the disclosed compounds of the present invention can be prepared according to one or more of these synthetic routes or specific examples, or via modifications thereof accessible to the person of ordinary skill in the art. US2013197033 (A1) 2013 Aug. 1; Segura, M. et al. Bioorg Chem. 2003, 31, 248; Peshkova, M. et al. Electroanalysis, 2010, 22, 19, 2147; Swanson, D. et al. Bioorg. Med. Chem. Lett, 2005, 16, 897; WO0107036A1; WO2011070592A2; German, N. et al./Bioorg. Med. Chem. Lett. 2008, 18 1368; WO2017040545 A1 2017 Mar. 9; WO2018026866 A1 2018 Feb. 8; Aslam, S. N. et al. Tetrahedron 2006, 62, 4214.

### Intermediate 1: tert-butyl (3S,4R)-4-(4-fluorophenyl)-3-(((methylsulfonyl)oxy)methyl) piperidine-1-carboxylate

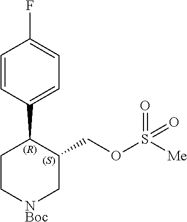

To a stirring solution of commercially available tert-butyl (3S,4R)-4-(4-fluorophenyl)-3-(hydroxymethyl) piperidine-1-carboxylate (3.0 g, 9.7 mmol) in dichloromethane (40 mL) at 0° C., triethylamine (2.7 mL, 19 mmol) was added followed by methanesulfonyl chloride (0.9 mL, 12 mmol). The solution was stirred at 0° C. for one hour. Upon completion, dichloromethane (50 mL) was added. The solution was extracted with hydrochloric acid (IN, 2×50 mL), water (50 mL), and brine (50 mL), dried over sodium sulfate. The solution was filtered and concentrated to give the title compound tert-butyl (3S,4R)-4-(4-fluorophenyl)-3-(((methylsulfonyl)oxy) methyl) piperidine-1-carboxylate (4.5 g, 9.2 mmol, 95% yield) as colorless oil. MS, ES⁺ m/z [M+Na]⁺=410.0. ¹H NMR (Chloroform-d) δ: 7.12-7.20 (m, 2H), 7.00-7.08 (m, 2H), 3.98 (dd, J=10.0, 2.9 Hz, 1H), 3.83 (dd, J=10.0, 6.7 Hz, 1H), 2.88-2.94 (m, 3H), 2.64-2.86 (m, 2H), 2.57 (td, J=11.8, 3.9 Hz, 1H), 2.00-2.11 (m, 1H), 1.77-1.86 (m, 1H), 1.60-1.75 (m, 2H), 1.47-1.54 (m, 9H).

### Intermediate 2: Preparation of 4-(((3S,4R)-1-(tert-butoxycarbonyl)-4-(4-fluorophenyl)piperidin-3-yl)methoxy)benzoic acid

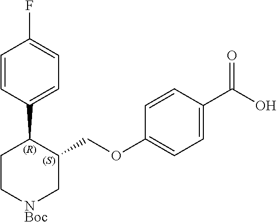

### Method A

### Step 1: Preparation of tert-butyl (3S,4R)-4-(4-fluorophenyl)-3-((4 (methoxycarbonyl)-phenoxy)methyl)piperidine-1-carboxylate

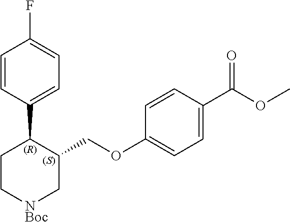

To a stirring solution of methyl 4-hydroxybenzoate (0.38 g, 2.5 mmol) in dimethylformamide (10 mL), sodium hydride (99 mg, 2.5 mmol) was added. The solution was stirred at room temperature for 10 min. Intermediate 1: tert-butyl (3S,4R)-4-(4-fluorophenyl)-3-(((methylsulfonyl)oxy) methyl) piperidine-1-carboxylate (1.0 g, 2.1 mmol) in dimethylformamide (10 mL) was added. The solution was stirred at room temperature for 24 hours. Ethyl acetate (80 mL) was added. The solution was washed with water (3×30 mL), then brine (30 mL). The organic phase was dried over sodium sulfate. The solution was filtered and concentrated. The residue was purified by normal phase chromatography using ethyl acetate/heptane (0-40%) to give the title compound as colorless oil 466 mg, 51% yield. TLC (Petroleum ether/Ethyl acetate=3/1, R_{f}=0.50). ¹H NMR (Chloroform-d) δ:7.90-8.00 (m, 3H), 7.15 (dd, J=8.5, 5.4 Hz, 2H), 6.95-7.04 (m, 2H), 6.85-6.93 (m, 1H), 6.72-6.79 (m, 2H), 3.86-3.93 (m, 4H), 3.70-3.78 (m, 1H), 3.60 (dd, J=9.5, 6.7 Hz, 1H), 2.84 (d, J=9.6 Hz, 2H), 2.71 (td, J=11.7, 3.8 Hz, 1H), 2.08 (d, J=10.9 Hz, 1H), 1.69-1.88 (m, 2H), 1.61-1.68 (m, 1H), 1.52 (s, 9H).

### Step 2: Preparation 4-(((3S,4R)-1-(tert-butoxycarbonyl)-4-(4-fluorophenyl)piperidin-3-yl)methoxy)benzoic acid

To a round bottom flask charged with tetrahydrofuran (5.0 mL) was added tert-butyl (3S,4R)-4-(4-fluorophenyl)-3-((4-(methoxycarbonyl)phenoxy)methyl)piperidine-1-carboxylate (1.20 g, 2.71 mmol, 1.0 eq) followed by addition of sodium hydroxide (541 mg, 13.5 mmol, 5.0 eq) as a solution in water (2.67 g, 148 mmol, 2.67 mL, 54.7 eq). Stirred at 40° C. for 12 hours. The reaction was monitored by LCMS. Upon completion, the crude reaction was added to water (10.0 mL). The pH was adjusted to 1. Extracted with ethyl acetate (5.0 mL×3). Washed the organic layer with brine (100 mL×3). Dried over with sodium sulfate. Concentrated the organic layer in vacuo. Used in next step without further purification. Title compound was obtained as a white solid (1.10 g, 2.56 mmol, 94.6% yield). MS, ES m/z [M−H]⁻=428.2.

### LI-900 Example 1:4-(((3S,4R)-4-(4-fluorophenyl)piperidin-3-yl)methoxy)benzoic acid hydrochloride

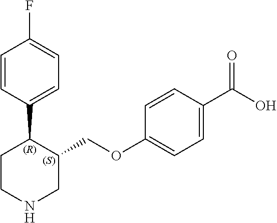

### Method B

To a stirring solution of 4-(((3S,4R)-1-(tert-butoxycarbonyl)-4-(4-fluorophenyl)piperidin-3-yl)methoxy)benzoic acid (180 mg, 0.42 mmol) in dichloromethane (3.0 mL) at room temperature, trifluoroacetic acid (3.0 mL) was added. The solution was stirred at room temperature for 1 hour. The solution was concentrated under reduced pressure. The residue was purified by automated reverse phase chromatography (5-95% acetonitrile/water; 0.05% TFA buffer). The TFA salt was converted to the hydrochloride salt by treating with HCl in diethyl ether (1 N, 20 ml) to give the title compound 4-(((3S,4R)-4-(4-fluorophenyl)piperidin-3-yl)methoxy)benzoic acid hydrochloride (58 mg, 0.18 mmol, 42% yield). MS, ES⁺ m/z [M+H]⁺=330.2. ¹H NMR (DMSO-d6) δ: 12.66 (br. s., 1H), 9.13 (br. s., 2H), 7.83 (d, J=8.8 Hz, 2H), 7.22-7.30 (m, 2H), 7.12-7.21 (m, 2H), 6.78-6.95 (m, 2H), 3.69-3.83 (m, 2H), 3.60-3.70 (m, 1H), 3.31-3.59 (m, 3H), 2.83-3.08 (m, 3H), 1.95-2.09 (m, 1H), 1.88 (d, J=14.1 Hz, 1H).

The following compounds 2-4 were synthesized according to Method A followed by Method B.

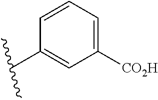 [SMILES: *c1cccc(C(=O)O)c1]

| Example | Name | R₁ | Analytical Data | Preparation Information |
|---|---|---|---|---|
| 2 | 3-(((3S,4R)-4-(4- fluorophenyl)piper- idin-3-yl)methoxy)- benzoic acid hydro- chloride | 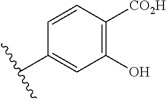 [SMILES: *c1ccc(C(=O)O)c(O)c1] | MS, ES⁺ m/z [M + H]⁺ = 330.0. ¹H NMR (DMSO- d6) δ: 13.02 (br. s., 1H), 9.24 (br. s., 2H), 7.47-7.53 (m, 1H), 7.32-7.42 (m, 1H), 7.20-7.31 (m, 3H), 7.10-7.21 (m, 2H), 7.00- | Method A using methyl 3- hydroxybenzoate (377 mg, 2.37 mmol) and interme- diate 2 (1.0 g, 2.06 mmol) followed by Method B to give 93 mg, 0.28 mmol, 87% yield. |
| | | | 7.07 (m, 1H), 3.68-3.78 | |
| | | | (m, 1H), 3.64 (dd, J = 9.9, | |
| | | | 6.3 Hz, 1H), 3.53 (d, J = | |
| | | | 11.6 Hz, 1H), 3.37 (d, J = | |
| | | | 12.1 Hz, 1H), 2.85-3.07 | |
| | | | (m, 3H), 2.56 (br. s., 1H), | |
| | | | 1.98-2.14 (m, 1H), 1.87 | |
| | | | (d, J = 12.6 Hz, 1H) | |
| 3 | 4-(((3S,4R)-4-(4- fluorophenyl)piper- idin-3-yl)methoxy)- 2-hydroxybenzoic acid hydrochloride | 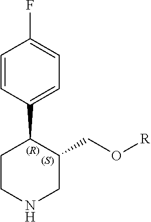 | MS, ES⁺ m/z [M + H]⁺ = 346.0. ¹H NMR (DMSO- d6) δ: 13.54-13.78 (m, 1H), 11.47 (br. s., 1H), 9.06-9.28 (m, 2H), 7.66 (d, J = 8.8 Hz, 1H), 7.10- 7.32 (m, 4H), 6.38 (dd, J = 8.8, 2.3 Hz, 1H), 6.30 (d, J = 2.3 Hz, 1H), 3.68- 3.77 (m, 1H), 3.59-3.68 (m, 2H), 3.51 (d, J = 11.6 Hz, 2H), 3.38 (d, J = 10.6 Hz, 1H), 2.77-3.06 (m, 3H), 1.95-2.12 (m, 1H), | Method A using methyl 3 methyl 4-hydroxy-2- methoxybenzoate (961 mg, 5.28 mmol) and interme- diate 2 (2.1 g, 4.40 mmol) followed by Method B to give 69 mg, 0.20 mmol, 46% yield. |
| | | | 1.87 (d, J = 13.4 Hz, 1H) | |
| 4 | 5-(((3S,4R)-4-(4- fluorophenyl)piper- idin-3-yl)methoxy)- 2-methoxybenzoic acid hydrochloride | 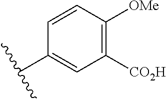 [SMILES: *c1ccc(OC)c(C(=O)O)c1] | MS, ES⁺ m/z [M + H]⁺ = 360.2. ¹H NMR (DMSO- d6) δ: 9.19-9.42 (m, 2H), 7.58-7.70 (m, 1H), 7.13- 7.31 (m, 5H), 6.45-6.53 (m, 1H), 6.38 (dd, J = 8.6, 2.3 Hz, 1H), 3.79-3.94 | Method A using methyl 5- hydroxy-2-methoxyben- zoate and intermediate 2 followed by Method B to give 80 mg, 0.22 mmol, 61% yield. |
| | | | (m, 1H), 3.72-3.78 (m, | |
| | | | 3H), 3.62-3.70 (m, 1H), | |
| | | | 3.30-3.56 (m, 2H), 2.71- | |
| | | | 3.04 (m, 3H), 2.55 (br. s., | |
| | | | 1H), 1.93-2.15 (m, 1H), | |
| | | | 1.88 (d, J = 11.9 Hz, 1H) | |

### LI-2475 Example 5: N-(2-aminoethyl)-4-(((3S,4R)-4-(4-fluorophenyl)piperidin-3-yl)-methoxy)benzamide hydrochloride

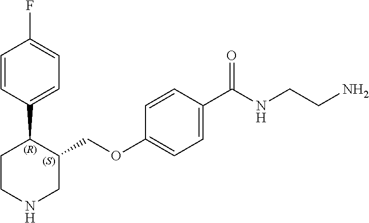

### Method C

### Step 1: Preparation of tert-butyl (3S,4R)-3-((4-((2-((tert-butoxycarbonyl)amino-)-ethyl)carbamoyl)phenoxy)methyl)-4-(4-fluorophenyl)piperidine-1-carboxylate

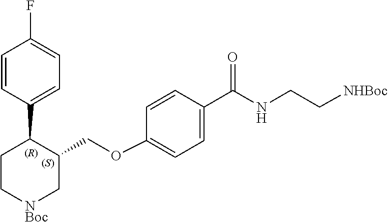

Intermediate 2: 4-(((3S,4R)-1-(tert-butoxycarbonyl)-4-(4-fluorophenyl)piperidin-3-yl)methoxy)benzoic acid (150 mg, 349 μmol, 1.0 eq) was added to a one necked round bottom flask charged with dimethylformamide (2.00 mL) at 15° C. Followed by addition of N-[(Dimethylamino)-1H-1,2,3-triazolo-[4,5-b]pyridin-1-ylmethylene]-N-methylmethanaminium hexafluorophosphate N-oxide HATU (199 mg, 523 μmol, 1.5 eq), triethylamine (53.0 mg, 523 μmol, 72.9 μL, 1.5 eq) and 4-dimethylaminopyridine DMAP (106 mg, 873 μmol, 2.5 eq) at 15° C. Stirred at 15° C. for 0.5 hr. Tert-butyl (2-aminoethyl)carbamate (83.9 mg, 523 μmol, 82.2 μL, 1.50 eq) was added to the solution mixture at 15° C. and stirred for 12 hrs. The reaction was monitored by LCMS. Upon completion, water was added to the crude reaction. Extracted with ethyl acetate (10.0 mL×3). Dried over sodium sulfate. Concentrated in vacuo. The residue was purified by prep-TLC (SiO₂, dichloromethane/methanol=5/1) to give tert-butyl (3S,4R)-3-((4-((2-((tert-butoxycarbonyl)amino)ethyl)carbamoyl)phenoxy)methyl)-4-(4-fluorophenyl)-piperidine-1-carboxylate (70 mg, 122 μmol, 35.0% yield) as yellow oil. MS, ES⁺ m/z [M+Na]⁺=594.5. ¹H NMR (400 MHZ, MeOD-d4) δ 7.72 (d, J=8.4 Hz, 2H), 7.25 (t, J=5.2 Hz 2H), 7.01 (t, J=8.8 Hz, 2H), 6.80 (d, J=8.8 Hz, 2H), 4.44 (d, J=12.8 Hz, 1H), 4.21 (d, J=13.4 Hz 1H), 4.12-4.07 (m, 1H), 3.75-3.73 (m, 1H), 3.66-3.59 (m, 2H), 3.41 (t, J=6.0 Hz 2H), 3.24 (t, J=6.0 Hz, 2H), 2.81-2.77 (m, 3H), 2.15-2.09 (m, 1H), 1.49 (s, 9H), 1.40 (s, 9H), 1.29-1.18 (m, 3H).

### Method D

### Step 2: Preparation of N-(2-aminoethyl)-4-(((3S,4R)-4-(4-fluorophenyl)piperidin-3-yl)methoxy)benzamide

Tert-butyl (3S,4R)-3-((4-((2-((tert-butoxycarbonyl)amino)ethyl)carbamoyl)-phenoxy)-methyl)-4-(4-fluorophenyl)piperidine-1-carboxylate (100 mg, 174 μmol, 1.0 eq) was added into a flask charged with HCl in 1,4-dioxane (2.0 M, 2.0 mL) at 25° C. The reaction was stirred for 2 hours at 25° C. The reaction was monitored by LCMS. Upon completion, the crude reaction was concentrated in vacuo. The residue was purified by prep-TLC (SiO₂, dichloromethane/methanol=5/1) to give the title compound N-(2-aminoethyl)-4-(((3S,4R)-4-(4-fluorophenyl)piperidin-3-yl)methoxy)benzamide hydrochloride (30.0 mg, 80.7 μmol, 46.1% yield) as white solid. MS, ES⁺ m/z [M+H]⁺=372.3. ¹H NMR 400 MHz, MeOD-d4) δ 7.79 (d, J=8.4 Hz, 2H), 7.30-7.27 (M, 2H), 7.05 (T, J=8.8 Hz, 2H), 6.87 (d, J=8.8 Hz, 2H), 3.84-3.80 (m, 1H), 3.74-3.68 (m, 2H), 3.62 (s, 1H), 4.06 (t, J=5.6 Hz 2H), 3.55-3.51 (m, 1H), 3.23-3.12 (m, 4H), 3.05-2.99 (m, 1H), 2.51-2.42 (m, 1H), 2.11-1.99 (m, 2H).

The following compounds 6-9 were synthesized according to Method C followed by Method D.

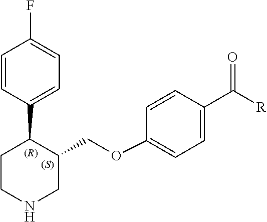

| Example | Name | R₁ | Analytical Data | Preparation Information |
|---|---|---|---|---|
| 6 | N-(2-(dimethylamino)- ethyl)-4-(((3S,4R)-4-(4- fluorophenyl)piperidin- 3-yl)methoxy)benzamide hydrochloride | 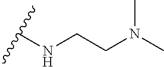 [SMILES: *NCCN(C)C] | MS, ES⁺ m/z [M + H]⁺ = 400.3. ¹H NMR (400 MHz, MeOD-d4) δ 7.82 (d, J = 8.8 Hz, 2H), 7.31- 7.27 (m, 2H), 8.35 (d, J = | Method C using N¹,N¹- dimethylethane-1,2-di- amine (46 mg, 523 μmol) and intermediate 2 (150 mg, 349 μmol) followed |
| | | | 7.6 Hz, 1H), 7.06 (t, J = | by Method D to give 20 |
| | | | 8.8 Hz, 2H), 6.88 (d, J = | mg, 50 μmol, 36% yield. |
| | | | 8.8 Hz, 2H) 3.84-3.81 | |
| | | | (m, 1H), 3.75-3.71 (m, | |
| | | | 4H), 3.56-3.52 (m, 1H), | |
| | | | 3.33 (t, J = 4.8 Hz, 2H), | |
| | | | 3.24-3.15 (m, 2H), 3.06- | |
| | | | 2.99 (m, 1H), 2.97 (s, | |
| | | | 6H), 2.53-2.43 (m, 1H), | |
| | | | 2.09-2.03 (m, 2H). | |
| 7 | N-(2-(dimethylamino)- ethyl)-4-(((3S,4R)-4-(4- fluorophenyl)piperidin- 3-yl)methoxy)-N-meth- ylbenzamide hydro- chloride | 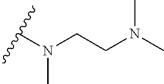 [SMILES: *N(C)CCN(C)C] | MS, ES⁺ m/z [M + H]⁺ = 414.1.¹HNMR (400 MHz, MeOD-d4) δ 7.46 (d, J = 8.4 Hz, 2H), 7.30- 7.28 (m, 2H), 7.06 (t, J = 8.8 Hz, 2H), 6.88 (d, J = | Modified Method C using N¹,N¹,N²-trimeth- ylethane-1,2-diamine (43 mg, 420 μmol), HOBt (57 mg, 419 μmol, 1.5 eq), TEA (57 mg, 558 μmol, |
| | | | 8.4 Hz, 2H), 3.87 (t, J = | 78 μL, 2.00 eq), EDCI |
| | | | 5.6 Hz, 2H), 3.83-3.80 | (80 mg, 419 μmol, 1.5 |
| | | | (m, 1H), 3.74-3.68 (m, | eq), DMAP (51 mg, 419 |
| | | | 2H), 3.55 (m, J = 12.8 | μmol, 1.5 eq) and inter- |
| | | | Hz, 1H), 3.45 (t, J = 6.0 | mediate 2 (120 mg, 279 |
| | | | Hz, 2H), 3.25-3.15 (m, | μmol) followed by |
| | | | 2H), 3.08 (s, 3H), 3.06- | Method D to give 20 |
| | | | 3.03 (m, 1H), 3.00 (s, | mg, 43 μmol, 32% yield. |
| | | | 6H), 2.54-2.47 (m, 1H), | |
| | | | 2.10-2.05 (m, 2H). | |
| 8 | N-(2-((diaminomethyl- ene)amino)ethyl)-4- (((3S,4R)-4-(4-fluoro- phenyl)piperidin-3-yl)- methoxy)benzamide | 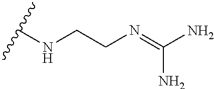 [SMILES: *NCCN=C(N)N] | MS, ES⁺ m/z [M + H]⁺ = 414.2. ¹H NMR(400 MHz, MeOD-d4) δ 7.79 (d, J = 8.8 Hz, 2H), 7.32- 7.28 (m, 2H), 7.05 (t, J = 8.8 Hz, 2H), 6.86 (d, J = | Modified Method C using 2-(2-aminoethyl)- 1,3-di-Boc-guanidine (126 mg, 419 μmol), HOBt (57 mg, 419 μmol, 1.5 eq), TEA (57 mg, 558 |
| | | | 8.4 Hz, 2H), 3.81 (dd, | μmol, 78 μL, 2.0 eq), |
| | | | J = 2.8 Hz, J = 10.0 Hz, | EDCI (80 mg, 419 μmol, |
| | | | 1H), 3.73-3.71 (m, 2H), | 1.5 eq), DMAP (51 mg, |
| | | | 3.55-3.51 (m, 3H), 3.41- | 419 μmol, 1.5 eq) and |
| | | | 3.38 (m, 2H), 3.21-3.15 | intermediate 2 (120 mg, |
| | | | (m, 2H), 3.06-2.99 (m, | 279 μmol) followed by |
| | | | 1H), 2.55-2.49 (m, 1H), | Method D to give 34 |
| | | | 2.16-2.04 (m, 2H). | mg, 75 μmol, 54% yield. |
| 9 | 4-(((3S,4R)-4-(4-fluoro- phenyl)piperidin-3-yl)- methoxy)-N-(2-ureido- ethyl)benzamide hydro- chloride | 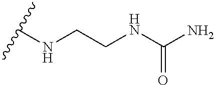 [SMILES: *NCCNC(N)=O] | MS, ES⁺ m/z [M + H]⁺ = 415.2. ¹H NMR (400 MHz, MeOD-d4) δ 7.76 (d, J = 8.8 Hz, 2H), 7.31- 7.28 (m, 2H), 7.06 (t, J = 8.8 Hz, 2H), 6.86 (d, J = | Method C using 1-(2- aminoethyl)urea (36 mg, 349 μmol), and interme- diate 2 (100 mg, 232 μmol) followed by Method D to give 20 |
| | | | 8.8 Hz, 2H), 3.81-3.78 | mg, 42 μmol, 24% yield. |
| | | | (m, 1H), 3.71-3.65 (m, | |
| | | | 2H), 3.53-3.46 (m, 3H), | |
| | | | 3.38-3.33 (m, 2H), 3.21- | |
| | | | 3.13 (m, 2H), 3.04-2.97 | |
| | | | (m, 1H), 2.51-2.43 (m, | |
| | | | 1H), 2.08-2.01 (m, 2H). | |

Preparation of phenol intermediates 3-7:

### Intermediate 3: tert-butyl (2-(4-hydroxyphenoxy)ethyl)carbamate

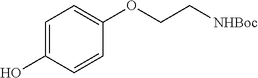 [SMILES: CC(C)(C)OC(=O)NCCOc1ccc(O)cc1]

### Method E

### Step 1: Preparation of tert-butyl (2-(4-(benzyloxy)phenoxy)ethyl)carbamate

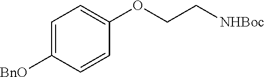 [SMILES: CC(C)(C)OC(=O)NCCOc1ccc(OCc2ccccc2)cc1]

4-(benzyloxy)phenol (5.00 g, 24.9 mmol, 1.0 eq) was added to a round bottom flask charged with acetonitrile (35.0 mL) followed by addition of tert-butyl (2-bromoethyl)carbamate (8.39 g, 37.4 mmol, 1.5 eq) and potassium carbonate (13.8 g, 99.8 mmol, 4.0 eq) to the solution mixture. The reaction was stirred at 50° C. for 12 hrs. The reaction was monitored by LCMS. Upon completion, the solids were filtered off from the crude reaction. Concentrated the filtrate in vacuo. Used without further purification to give tert-butyl (2-(4-(benzyloxy)phenoxy)ethyl)carbamate (10.1 g, 19.3 mmol, 77.4% yield, 65.2% purity by LCMS) as pale yellow solid. MS, ES⁺ m/z [M+Na]⁺=366.2.

### Method F

### Step 2: Preparation of intermediate 3: tert-butyl (2-(4-hydroxyphenoxy)ethyl)carbamate

Ethanol (20.0 mL) was added to round bottom flask charged with and palladium on carbon (300 mg, 1.35 mmol, 0.15 eq) followed by addition of tert-butyl (2-(4-(benzyloxy)phenoxy)ethyl)carbamate (1.00 g, 2.91 mmol, 1.0 eq) to the mixture. Degassed the mixture with hydrogen gas three times. Stirred the reaction mixture at 15° C. for 12 hrs. The reaction was monitored by LCMS. Upon completion, the crude reaction was filtered through celite. The filtrate was collected. The crude product was purified by column chromatography (SiO₂, Petroleum ether/Ethyl acetate=10/1 to 2/1) to give tert-butyl (2-(4-hydroxyphenoxy)ethyl)carbamate (2.83 g, 6.76 mmol, 77.3% yield, 60.5% purity by LCMS, black solid. MS, ES⁺ m/z [M+Na]⁺=276.2.

The following intermediates 4-6 were synthesized according to Method E followed by Method F.

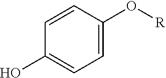

| Inter- | | | | |
|---|---|---|---|---|
| mediate | Name | R₁ | Analytical Data | Preparation Information |
| 4 | 4-(2-(dimethylamino)- ethoxy)phenol | 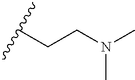 [SMILES: *CCN(C)C] | MS, ES⁺ m/z [M + H]⁺ = | Modified Method E using 4- (benzyloxy)phenol (2.00 g, 9.99 mmol, 1.00 eq), 2- chloro-N,N-dimethylethan- 1-amine (1.29 g, 11.9 mmol, |
| | | | | 1.20 eq), sodium hydride (599 |
| | | | | mg, 60.0% purity 1.5 eq), |
| | | | | DMF (2 mL), 90° C. followed |
| | | | | by modified Method F using |
| | | | | methanol (4 mL), stirring at |
| | | | | 25° C. to give 650 mg crude. |
| 5 | 4-(3-(dimethylamino)- propoxy)phenol | 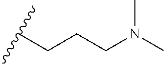 [SMILES: *CCCN(C)C] | MS, ES⁺ m/z [M + H]⁺ = | Method E using 4-(benzyl- oxy)phenol (1.00 g, 4.99 mmol, 1.0 eq), 3-chloro-N,N- dimethylpropan-1-amine (728 |
| | | | | mg, 5.99 mmol, 1.2 eq), |
| | | | | followed by modified Method |
| | | | | F using methanol (4 mL), |
| | | | | stirring at 25° C. to give 750 |
| | | | | mg crude. |
| 6 | tert-butyl 2-(4-hydroxy- phenoxy)acetate | 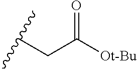 [SMILES: *CC(=O)OC(C)(C)C] | MS, ES⁺ m/z [M + H]⁺ = 247.1 | Method E using 4-(benzyl- oxy)phenol (1.00 g, 4.99 mmol, 1.0 eq), tert-butyl 2- bromoacetate (1.46 g, 7.49 mmol, 1.11 mL, 1.5 eq), |
| | | | | followed by modified Method |
| | | | | F using methanol (4 mL), |
| | | | | stirring at 25° C. to 47% yield. |

Preparation of phenol intermediates 7-10:

### Intermediate 7:2-(4-(benzyloxy)phenoxy)ethan-1-ol

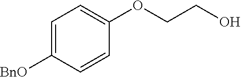 [SMILES: OCCOc1ccc(OCc2ccccc2)cc1]

The title intermediate was prepared according to modified Method E using 4-(benzyloxy)phenol (5.00 g, 24.9 mmol, 1.0 eq), dimethylformamide (25 mL), 2-bromoethan-1-ol (3.43 g, 27.4 mmol, 1.95 mL, 1.1 eq) to give 4.40 g as a brown solid. MS, ES⁺ m/z [M+H]⁺=245.1. ¹H NMR (400 MHz, CDCl₃) δ 7.45-7.33 (m, 5H), 6.94-6.92 (m, 2H), 6.91-6.86 (m, 2H), 5.03 (s, 2H), 4.05-4.03 (m, 2H), 3.96-3.92 (m, 2H), 2.13-2.10 (t, J=5.6 Hz, 1H).

### Intermediate 8:4-(2-((tetrahydro-2H-pyran-2-yl)oxy)ethoxy)phenol

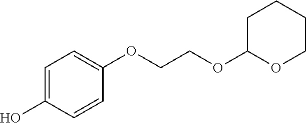 [SMILES: Oc1ccc(OCCOC2CCCCO2)cc1]

### Method G

### Step 1: Preparation of 2-(2-(4-(benzyloxy)phenoxy)ethoxy)tetrahydro-2H-pyran

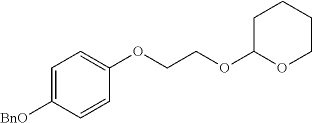 [SMILES: c1ccc(COc2ccc(OCCOC3CCCCO3)cc2)cc1]

Intermediate 7 2-(4-(benzyloxy)phenoxy)ethan-1-ol (1.00 g, 4.09 mmol, 1.0 eq) was taken up in dichloromethane (6 mL). Pyridine p-toluenesulfonate (102 mg, 409 μmol, 0.1 eq) was added to the solution mixture followed by 3,4-dihydro-2H-pyran (516 mg, 6.14 mmol, 561 μL, 1.5 eq). Stirred the reaction mixture at 15° C. for 12 hrs. The reaction was monitored by LCMS. Upon completion, water (20 mL) was added to the crude reaction. Extracted with dichloromethane (20 mL×3). Washed the organic layer with brine (100 mL×3). Dried over with sodium sulfate. Concentrated the organic layer in vacuo. Used in next step without further purification. Title compound was obtained (1.57 g, crude) as brown oil. MS, ES⁺ m/z [M+H]⁺=351.2.

### Step 2: Preparation of 4-(2-((tetrahydro-2H-pyran-2-yl)oxy)ethoxy)phenol

The title intermediate was prepared according to modified Method F using 2-(2-(4-(benzyloxy)phenoxy)ethoxy)tetrahydro-2H-pyran (1.47 g, 4.48 mmol, 1.0 eq), methanol (10.2 mL) to give 995 mg, purity) as pale yellow oil. 1H NMR (400 MHZ, CDCl3) δ 6.84-6.80 (m, 2H), 6.77-6.47 (m, 2H), 4.71 (t, J=3.6 Hz, 1H), 4.64 (s, 1H), 4.12-4.00 (m, 3H), 3.94-3.82 (m, 1H), 3.80-3.57 (m, 1H), 3.57-3.51 (m, 1H), 1.88-1.71 (m, 2H), 1.68-1.51 (m, 4H).

### Intermediate 9:4-(2-(2-((tetrahydro-2H-pyran-2-yl)oxy)ethoxy)ethoxy)phenol

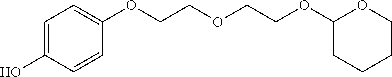 [SMILES: Oc1ccc(OCCOCCOC2CCCCO2)cc1]

### Step 1: Preparation of 2-(2-bromoethoxy)tetrahydro-2H-pyran

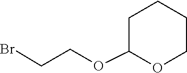

The title intermediate was prepared according to modified Method G using 2-bromoethan-1-ol (25.0 g, 200 mmol, 14.2 mL, 1.0 eq), 3,4-dihydro-2H-pyran (25.2 g, 300 mmol, 27.4 mL, 1.5 eq) and pyridine p-toluenesulfonate (5.03 g, 20.01 mmol, 0.10 eq). The crude product was purified by silica gel column chromatography (SiO2, Petroleum ether/Ethyl acetate=100/1 to 20/1) to give 40.0 g, 172 mmol, 86.0% yield, ˜90.0% TLC purity as colourless oil. TLC (Petroleum ether/Ethyl acetate=20/1, product: R_{f}=0.55)

### Step 2: Preparation of 2-(2-(2-(4-(benzyloxy)phenoxy)ethoxy)ethoxy)tetrahydro-2H-pyran

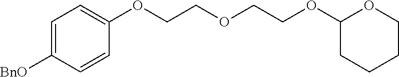 [SMILES: c1ccc(COc2ccc(OCCOCCOC3CCCCO3)cc2)cc1]

Intermediate 7:2-(4-(benzyloxy)phenoxy)ethan-1-ol (1.00 g, 4.09 mmol, 1.0 eq) was taken into water (7.00 mL). To this solution was added 2-(2-bromoethoxy)tetrahydro-2H-pyran (3.42 g, 16.3 mmol, 2.48 mL, 4.0 eq), sodium hydroxide (2.62 g, 65.5 mmol, 16 eq) and tetrabutylammonium bisulfate (13.90 mg, 40.94 μmol, 0.01 eq). The reaction was stirred at 65° C. for 12 hrs and monitored by TLC (Petroleum ether/Ethyl acetate=1/2, product: R_{f}=0.70). The organic phase was extracted methyl tert-butylether (5.0 mL×3) and washed with brine (5.0 mL×2), dried over sodium sulfate and concentrated in vacuo. The crude product was purified by silica gel column chromatography (SiO₂, Petroleum ether/Ethyl acetate=50/1 to 10/1) to give 2-(2-(2-(4-(benzyloxy)phenoxy)ethoxy)ethoxy)tetrahydro-2H-pyran (1.28 g, ˜90% TLC purity) as pale yellow oil.

### Step 3: Preparation of 4-(2-(2-((tetrahydro-2H-pyran-2-yl)oxy)ethoxy)ethoxy)phenol

The title intermediate was prepared according to modified Method F using 2-(2-(2-(4-(benzyloxy)phenoxy)ethoxy)ethoxy)tetrahydro-2H-pyran (1.18 g, 3.17 mmol, 1.0 eq), methanol (8.3 mL) to give 810 mg, yellow oil. MS, ES⁺ m/z [M+Na]⁺=305.2. ¹H NMR (400 MHz, CDCl3) δ 6.80-6.73 (m, 4H), 4.83 (s, 1H), 4.66-4.65 (m, 1H), 4.07-4.05 (m, 2H), 3.93-3.83 (m, 4H), 3.76-3.68 (m, 2H), 3.62-3.54 (m, 1H), 3.54-3.49 (m, 1H), 1.88-1.49 (m, 7H).

### Intermediate 10: tert-butyl 2-(2-(4-hydroxyphenoxy)ethoxy)acetate

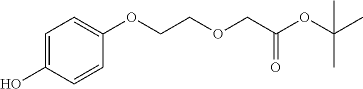 [SMILES: CC(C)(C)OC(=O)COCCOc1ccc(O)cc1]

### Step 1: Preparation of tert-butyl 2-(2-(4-(benzyloxy)phenoxy)ethoxy)acetate

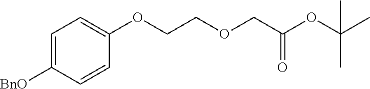 [SMILES: CC(C)(C)OC(=O)COCCOc1ccc(OCc2ccccc2)cc1]

Intermediate 7:2-(4-(benzyloxy)phenoxy)ethan-1-ol (2.00 g, 8.19 mmol, 1.0 eq) was taken up in toluene (10.0 mL). To the solution was added potassium hydroxide (1.39 g, 4.09 mmol, 0.50 eq) followed by drop wise addition of tert-butyl 2-bromoacetate (6.39 g, 32.7 mmol, 4.84 mL, 4.0 eq). To the reaction mixture was added a solution of potassium hydroxide (15 M, 16.3 mL, 30 eq) in water (16.3 mL). The reaction was stirred at 15° C. for 2 hrs and monitored by TLC (petroleum ether/ethyl acetate=2/1, R_{f}=0.55). Upon completion, ethyl acetate was added to the reaction mixture (20.0 mL×3). The organic phase was separated, washed with brine (20.0 mL×2). Dried over sodium sulfate, and concentrated. The crude product was purified by silica gel column chromatography (Petroleum ether/Ethyl acetate=5/1 to 0/1) to give tert-butyl 2-(2-(4-(benzyloxy)phenoxy)ethoxy)acetate (2.30 g, 6.10 mmol, 74% yield), white solid. 1H NMR (400 MHZ, CDCl3) δ 7.44-7.27 (m, 5H), 6.92-6.85 (m, 4H), 5.02 (s, 2H), 4.14-4.10 (m, 4H), 3.92-3.89 (t, J=4.8 Hz, 2H), 1.49 (s, 9H).

### Step 2: Preparation of tert-butyl 2-(2-(4-hydroxyphenoxy)ethoxy)acetate

The title intermediate was prepared according to modified Method F using tert-butyl 2-(2-(4-(benzyloxy)phenoxy)ethoxy)acetate (0.90 g, 2.51 mmol, 1.0 eq), methanol (5.0 mL) to give 800 mg, green solid. TLC (petroleum ether/ethyl acetate=2/1, R_{f}=0.40)

### Example 10: 2-(4-(((3S,4R)-4-(4-fluorophenyl)piperidin-3-yl)methoxy)phenoxy)-ethan-1-amine hydrochloride

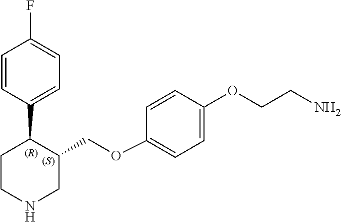

### Method H

### Step 1: Preparation of tert-butyl (3S,4R)-3-((4-(2-((tert-butoxycarbonyl)amino)ethoxy)phenoxy)methyl)-4-(4-fluorophenyl)piperidine-1-carboxylate

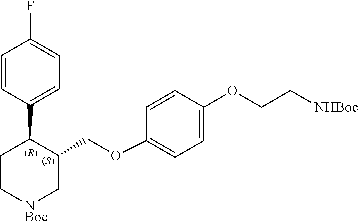

To a solution of intermediate 3 tert-butyl (2-(4-hydroxyphenoxy)ethyl)carbamate (156 mg, 619 μmol, 1.2 eq) in dimethylformamide (7.00 mL) was added sodium hydride (24.7 mg, 619 μmol, 60% purity, 1.2 eq) followed by addition of intermediate 1 tert-butyl (3S,4R)-4-(4-fluorophenyl)-3-(((methylsulfonyl)oxy)methyl) piperidine-1-carboxylate (200 mg, 516 μmol, 1.0 eq). The reaction was stirred at 50° C. for 12 hrs and was monitored by LCMS. Upon completion, the crude reaction was quenched with saturated ammonium chloride (50.0 mL) and filtered through celite and washed with ethyl acetate (50.0 mL, 2×). Collected the filtrate. The organic phase was separated and washed with brine (50.0 mL, 2×), dried over sodium sulfate and concentrate in vacuum. The crude product was purified by prep-TLC (SiO₂, PE/EA=I/O to 3/1) to give tert-butyl (3S,4R)-3-((4-(2-((tert-butoxycarbonyl)amino)ethoxy)phenoxy)methyl)-4-(4-fluorophenyl)piperidine-1-carboxylate (200 mg, 365 μmol, 70.7% yield) as colorless oil. MS, ES⁺ m/z [M+Na]⁺=567.3. ¹H NMR (400 MHz MeOH-d4) δ 7.22 (q, J=2.8 Hz 2H), 7.00 (t, J=8.8 Hz 2H), 6.60-6.79 (m, 4H), 4.19 (d, J=13.6 Hz, 1H), 3.89 (t, J=5.6 Hz, 1H), 3.60 (q, J=6.8 Hz 2H), 3.59 (d, J=2.4 Hz 1H), 3.50 (s, 1H), 3.35 (q, J=5.6 Hz 2H), 2.81 (q, J=37.6 Hz, 3H), 2.03 (t, J=4.40 Hz 1H), 1.69-1.76 (m, 2H), 1.48 (s, 9H), 1.42 (s, 9H), 1.17 (t, J=7.2 Hz 1H).

### Step 4: Preparation of 2-(4-(((3S,4R)-4-(4-fluorophenyl)piperidin-3-yl)methoxy)phenoxy)ethan-1-amine hydrochloride

The title compound was prepared according to modified Method D using tert-butyl (3S,4R)-3-((4-(2-((tert-butoxycarbonyl)amino)ethoxy)phenoxy)methyl)-4-(4-fluorophenyl)piperidine-1-carboxylate (150 mg, 275 μmol, 1.0 eq). The crude product was purified by prep-HPLC (column: Phenomenex Luna C18 100×30-mm×5-μm; mobile phase: [water (0.04% HCl)-ACN]; B %: 1%-20%, 10 min) to give 2-(4-(((3S,4R)-4-(4-fluorophenyl)-piperidin-3-yl)methoxy)phenoxy)ethan-1-amine (24.0 mg, 63.0 μmol, 22.8% yield) as white solid. MS, ES⁺ m/z [M+H]⁺=345.2. ¹H NMR (400 MHZ, MeOD-d4) δ 7.30 (q, J=3.2 Hz, 2H), 7.05 (t, J=7.2 Hz, 2H), 6.89 (d, J=8.8 Hz, 2H), 6.75 (t, J=2.0 Hz, 2H), 4.14 (t, J=5.2 Hz, 2H), 3.66-3.70 (m, 2H), 3.60 (d, J=6.4 Hz, 2H), 3.32 (d, J=6.0 Hz, 2H), 3.21 (d, J=48.4 Hz, 2H), 3.13 (d, J=22.4 Hz, 1H), 2.47 (d, J=2.80 Hz, 1H), 2.01-2.08 (m, 2H).

The following compounds 11-14 were synthesized according to Method E followed by

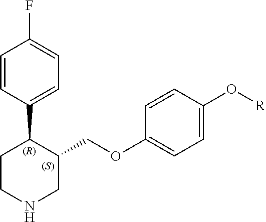

modified Method D.

| Example | Name | R₁ | Analytical Data | Preparation Information |
|---|---|---|---|---|
| 11 | 2-(4-(((3S,4R)-4-(4- fluorophenyl)piper- idin-3-yl)methoxy)- phenoxy)-N,N-di- methylethan-1-amine hydrochloride | 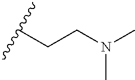 [SMILES: *CCN(C)C] | MS, ES⁺ m/z [M + H]⁺ = 373.2. ¹H NMR (400 MHz, MeOD-d4) δ 7.23- 7.25 (m, 2H), 6.98-7.02 (m, 2H), 6.72-6.85 (m, 2H), 6.61-6.70 (m, 2H), | Method H using inter- mediate 4 (56.1 mg, 309 μmol, 1.2 eq), and inter- mediate 1 (100 mg, 258 μmol, 1.0 eq) followed by Method D to give (107 |
| | | | 4.22-4.25 (m, 2H), 3.17- | mg, 287 μmol, 91% yield) |
| | | | 3.52 (m, 2H), 3.38-3.52 | |
| | | | (m, 4H), 2.91-3.18 (m, | |
| | | | 2H), 2.76-2.94 (m, 7H), | |
| | | | 2.25-2.81 (m, 1H), 1.79- | |
| | | | 2.08 (m, 2H). | |
| 12 | 3-(4-(((3S,4R)-4-(4- fluorophenyl)piper- idin-3-yl)methoxy)- phenoxy)-N,N-di- | 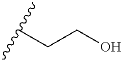 [SMILES: *CCO] | MS, ES⁺ m/z [M + H]⁺ = 387.2. ¹H NMR (400 MHz, MeOD-d4) δ 7.23- 7.38 (m, 2H), 6.98-7.02 | Method H using inter- mediate 5 (120 mg, 616 μmol, 1.2 eq), interme- diate 1 (200 mg, 516 |
| | methylpropan-1- | | (m, 2H), 6.72-6.85 (m, | μmol, 1.0 eq) followed by |
| | amine hydrochloride | | 2H), 6.61-6.70 (m, 2H), | Method D to give (96.0 |
| | | | 4.22-4.25 (m, 2H), 3.62- | mg, 248 μmol, 81% yield) |
| | | | 3.78 (m, 2H), 3.54-3.62 | |
| | | | (m, 2H), 3.3.17-3.52 (m, | |
| | | | 2H), 2.91-3.18 (m, 1H), | |
| | | | 2.76-2.94 (m, 7H), 2.25- | |
| | | | 2.81 (m, 1H), 2.22-2.34 | |
| | | | (m, 2H), 1.79-2.08 (m, | |
| | | | 2H). | |
| 13 | 2-(4-(((3S,4R)-4-(4- fluorophenyl)piper- idin-3-yl)methoxy)- phenoxy)ethan-1-ol | 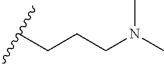 [SMILES: *CCCN(C)C] | MS, ES⁺ m/z [M + H]⁺ = 346.2. ¹H NMR (400 MHz, MeOD-d4) δ 7.30- 7.28 (m, 2H), 7.07 (t, J = | Method H using inter- mediate 8 (147 mg, 619 μmol, 1.2 eq), interme- diate 1 (200 mg, 516 |
| | hydrochloride | | 8.8 Hz, 2H), 6.83-6.82 | μmol, 1.0 eq) followed |
| | | | (d, J = 6.8 Hz, 2H), 6.72- | by Method D to give |
| | | | 6.69 (d, J = 9.2 Hz, 2H), | (40.0 mg, 101 μmol, |
| | | | 3.96-3.94 (m, 2H), 3.83- | 53.7% yield) |
| | | | 3.81 (m, 2H), 3.96-3.94 | |
| | | | (m, 2H),, 3.70-3.66 (m, | |
| | | | 2H), 3.20-3.13 (m, 2H), | |
| | | | 3.00-2.96 (m, 1H), 2.40- | |
| | | | 2.39 (m, 1H), 2.07-2.00 | |
| | | | (m, 5H). | |
| 14 | 2-(2-(4-(((3S,4R)-4- (4-fluorophenyl)- piperidin-3-yl) methoxy)phenoxy)- ethoxy)ethan-1-ol hydrochloride | 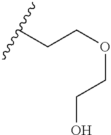 [SMILES: *CCOCCO] | MS, ES⁺ m/z [M + H]⁺ = 390.2. ¹H NMR (400 MHz, MeOD-d4) δ 7.30- 7.26 (m, 2H), 7.08-7.03 (t, J = 8.8 Hz, 2H), 6.82- 6.80 (d, J = 6.8 Hz, 2H), 6.71-6.69 (d, J = 9.2 Hz, 2H), 4.05-4.03 (m, 2H), 3.80-3.78 (m, 2H), 3.69- | Method H using inter- mediate 9 (166 mg, 619 μmol, 1.2 eq), interme- diate 1 (200 mg, 516 μmol, 1.0 eq) followed by Method D to give (55.0, 129 μmol, 72% yield) |
| | | | 3.66 (m, 4H), 3.61 (m, | |
| | | | 4H), 3.30 (m, 2H), 3.17- | |
| | | | 2.97 (m, 1H), 2.45-2.38 | |
| | | | (m, 1H), 2.06-2.02 (m, | |
| | | | 2H). | |

### Example 15:2-(4-(((3S,4R)-4-(4-fluorophenyl)piperidin-3-yl)methoxy)phenoxy)acetic acid hydrochloride

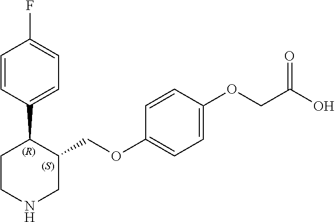

### Method I

### Step 1: Preparation of tert-butyl (3S,4R)-3-((4-(2-(tert-butoxy)-2-oxoethoxy)phenoxy)methyl)-4-(4-fluorophenyl)piperidine-1-carboxylate

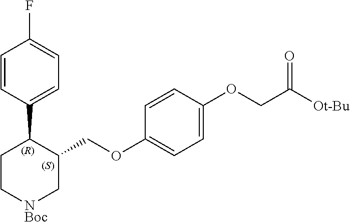

Intermediate 6 tert-butyl 2-(4-hydroxyphenoxy)acetate (0.20 g, 646 μmol, 1.0 eq) and tert-butyl (3S,4R)-4-(4-fluorophenyl)-3-(hydroxymethyl) piperidine-1-carboxylate (188 mg, 840 μmol, 1.3 eq) were taken up in tetrahydrofuran (4.00 mL). Triphenylphospine (254 mg, 969 μmol, 1.5 eq) was added to the solution mixture. A solution of 1,1′-(azodicarbonyl)dipiperidine (244 mg, 969 μmol, 1.5 eq) in tetrahydrofuran (1.00 mL) was added drop wise at 0° C. The reaction was stirred at 15° C. for 12 hrs and monitored by LCMS. Upon completion, the crude reaction was concentrated in vacuo then diluted with methanol (5.0 mL) and concentrated once again. The crude product was purified by silica gel column chromatography (SiO2 Petroleum ether/Ethyl acetate=2/1) to give tert-butyl (3S,4R)-3-((4-(2-(tert-butoxy)-2-oxoethoxy)phenoxy)methyl)-4-(4-fluorophenyl)-piperidine-1-carboxylate (150 mg, 290 μmol, 45.0% yield) as colorless oil. MS, ES⁺ m/z [M+Na]+=538.2.

### Step 2: Preparation of 2-(4-(((3S,4R)-4-(4-fluorophenyl)piperidin-3-yl)methoxy)phenoxy)acetic acid hydrochloride

The title compound was prepared according to modified Method D using tert-butyl (3S,4R)-3-((4-(2-(tert-butoxy)-2-oxoethoxy)phenoxy)methyl)-4-(4-fluorophenyl)piperidine-1-carboxylate (0.10 g, 193 μmol, 1.0 eq) to give (70 mg, 167 μmol, 87% yield). MS, ES⁺ m/z [M+H]⁺=360.1. ¹H NMR (400 MHZ, MeO-d4) δ 7.29-7.26 (m, 2H), 7.09 (t, J=8.4 Hz, 2H), 6.84-6.80 (m, 2H), 6.74-6.69 (m, 2H), 4.55 (s, 1H), 3.76-3.66 (m, 4H), 3.60-3.57 (m, 1H), 3.52 ((d, J=12.8 Hz, 1H), 3.20-3.13 (m, 2H), 2.98 (td, J=12.0 Hz, J=5.2 Hz, 1H), 2.42-2.38 (m, 1H), 2.09-1.99 (m, 2H).

### Example 16 2-(2-(4-(((3S,4R)-4-(4-fluorophenyl)piperidin-3-yl)methoxy)phenoxy)-ethoxy)acetic acid hydrochloride

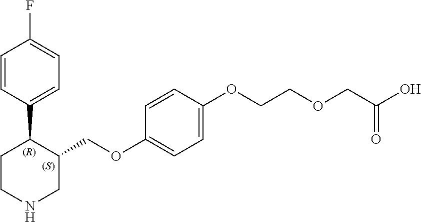

The title compound was prepared according to modified Method I using intermediate 10: tert-butyl 2-(2-(4-hydroxyphenoxy)ethoxy)acetate (0.30 g, 969 μmol, 1.0 eq), and tert-butyl (3S,4R)-4-(4-fluorophenyl)-3-(hydroxymethyl) piperidine-1-carboxylate (338 mg, 1.26 mmol, 1.3 eq) followed by Method D to give 2-(2-(4-(((3S,4R)-4-(4-fluorophenyl)piperidin-3-yl)methoxy)phenoxy)ethoxy)acetic acid hydrochloride 100 mg, 239 μmol, 67% yield, white solid. MS, ES⁺ m/z [M+H]⁺=404.2. ¹H NMR (400 MHZ, MeOD-d4) δ 7.31-7.26 (m, 2H), 7.09-7.04 (m, 2H), 6.84-6.80 (m, 2H), 6.72-6.68 (m, 2H), 4.17 (s, 2H), 4.07-4.05 (m, 2H), 3.87-3.85 (m, 2H), 3.68 (dd, J=2.8 Hz, J=10.0 Hz 2H), 3.66 (s, 2H), 3.58 (dd, J=2.8 Hz, J=10.0 Hz, 1H), 3.53 (d, J=12 Hz, 1H), 3.21-3.13 (m, 1H), 3.02-2.95 (m, 1H), 2.45-2.37 (m, 1H), 2.07-2.01 (m, 2H).

### Intermediate 11: (benzo[d][1,3]dioxol-5-ylmethyl)triphenylphosphonium bromide

The title intermediate was prepared according to the literature procedure by S. N. Aslam et al. Tetrahedron 2006, 62, 4214 using benzo[d][1,3]dioxol-5-ylmethanol 5.00 g, 32.8 mmol, 1.0 eq to give (10.0 g, 18.8 mmol, 81.0% yield, 90.0% purity) as white solid. TLC (Petroleum ether/Ethyl acetate=3/1, R_{f}=0.01). 1H NMR (400 MHZ, CDCl3) δ 7.76-7.69 (m, 9H), 7.63-7.60 (m, 6H), 6.60-6.57 (m, 1H), 6.51-6.49 (m, 2H), 5.29 (d, J=14 Hz, 2H), 0.37-0.41 (m, 2H).

### Example 17: (3S,4R)-3-(2-(benzo[d][1,3]dioxol-5-yl)ethyl)-4-(4-fluorophenyl)-1,1-dimethylpiperidin-1-ium iodide

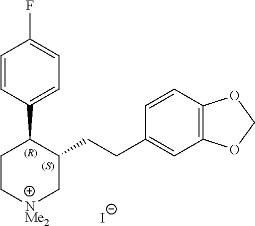

### Step 1: Preparation of tert-butyl (3S,4R)-4-(4-fluorophenyl)-3-formylpiperidine-1-carboxylate

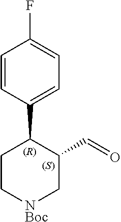

Oxalyl chloride (738 mg, 5.82 mmol, 509 μL, 1.2 eq) was added to a round bottom flask charged with dichloromethane (6.30 mL). Dimethylsulfoxide (909 mg, 11.6 mmol, 909 μL, 2.4 eq) was added drop wise to the mixture at −78° C. To the solution mixture was added tert-butyl (3S,4R)-4-(4-fluorophenyl)-3-(hydroxymethyl) piperidine-1-carboxylate (1.50 g, 4.85 mmol, 1.0 eq) in dichloromethane (3.00 mL) to the mixture. Stirred at −78° C. for 1.5 h. Triethylamine (1.23 g, 12.1 mmol, 1.69 mL, 2.50 eq) was added to the mixture. Stirred at −78° C. for 0.5 hr. Warmed to 15° C. and stirred for 1 hr. The reaction was monitored by TLC. TLC (Petroleum ether/Ethyl acetate=3/1, R_{f}=0.43) showed consumption of starting material. Upon completion, the crude reaction was quenched with ammonium chloride (50.0 mL). Extracted organic phase with ethyl acetate (20.0 mL×4), washed with brine, dried over sodium sulfate. Concentrated in vacuo. Took crude product to next step without purification. Obtained the title compound tert-butyl (3S,4R)-4-(4-fluorophenyl)-3-formylpiperidine-1-carboxylate (1.26 g, 4.10 mmol, 84.5% yield) as colorless oil.

### Step 2: Preparation of tert-butyl (3S,4R)-3-((E)-2-(benzo[d][1,3]dioxol-5-yl)vinyl)-4-(4-fluorophenyl)piperidine-1-carboxylate

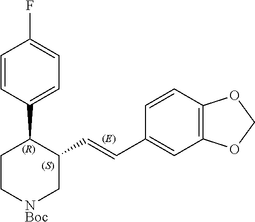

Dimethylsulfoxide (8.00 mL) was added to a round bottom flask followed by addition of sodium hydride (245 mg, 6.15 mmol, 60.0% purity, 1.5 eq) and (benzo[d][1,3]dioxol-5-ylmethyl)triphenylphosphonium bromide (2.35 g, 4.92 mmol, 1.2 eq). Stirred at 15° C. for 0.5 hr. Tert-butyl (3S,4R)-4-(4-fluorophenyl)-3-formylpiperidine-1-carboxylate (1.26 g, 4.10 mmol, 1.0 eq) was added to the reaction mixture and stirred at 15° C. for 1 hr. The reaction was monitored by LCMS. Upon completion, the crude reaction was quenched with ammonium chloride (20.0 mL). Extracted organic phase with ethyl acetate (20.0 mL×3), washed with brine, dried over sodium sulfate. Concentrated in vacuo. The crude product was purified by column chromatography (SiO₂, Petroleum ether/Ethyl acetate=I/O to 0/1) to give tert-butyl (3S,4R)-3-((E)-2-(benzo[d][1,3]dioxol-5-yl) vinyl)-4-(4-fluorophenyl)piperidine-1-carboxylate (0.85 g, 2.00 mmol, 48.7% yield) as yellow oil. MS, ES⁺ m/z [M+Na]⁺=448.3.

### Step 3: Preparation of tert-butyl (3S,4R)-3-(2-(benzo[d][1,3]dioxol-5-yl)ethyl)-4-(4-fluorophenyl)piperidine-1-carboxylate

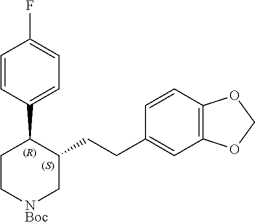

Methanol (4.2 mL) was added to a round bottom flask charged with wet palladium on carbon (0.30 g, 141 μmol, 10.0% purity) followed by addition of tert-butyl (3S,4R)-3-((E)-2-(benzo[d][1,3]dioxol-5-yl) vinyl)-4-(4-fluorophenyl)piperidine-1-carboxylate (0.85 g, 2.00 mmol, 1.0 eq) to the mixture. Degassed with hydrogen gas 3 times. Stirred at 30° C. for 3 hrs under hydrogen (50 Psi). The reaction was monitored by LCMS. Upon completion, the crude reaction was filtered to remove solids. Concentrated in vacuo. Took crude product to next step without purification. Obtained the title compound tert-butyl (3S,4R)-3-(2-(benzo[d][1,3]dioxol-5-yl)ethyl)-4-(4-fluorophenyl)piperidine-1-carboxylate (0.80 g, 1.87 mmol, 93.6% yield) as colorless oil. MS, ES⁺ m/z [M+Na]⁺=450.4.

### Step 4: Preparation of (3S,4R)-3-(2-(benzo[d][1,3]dioxol-5-yl)ethyl)-4-(4-fluorophenyl)piperidine hydrochloride

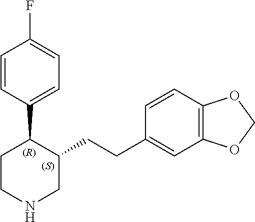

The title compound was prepared according to modified Method D using tert-butyl (3S,4R)-3-(2-(benzo[d][1,3]dioxol-5-yl)ethyl)-4-(4-fluorophenyl)piperidine-1-carboxylate (0.80 g, 1.87 mmol, 1.0 eq) to give (0.70 g, crude) as black oil. MS, ES⁺ m/z [M+H]⁺=328.3. Took crude product to next step without purification.

### Step 5: Preparation of (3S,4R)-3-(2-(benzo[d][1,3]dioxol-5-yl)ethyl)-4-(4-fluorophenyl)-1-methylpiperidine

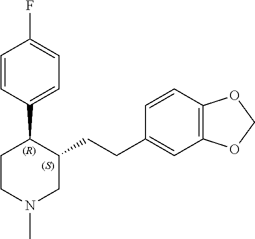

(3S,4R)-3-(2-(benzo[d][1,3]dioxol-5-yl)ethyl)-4-(4-fluorophenyl)piperidine hydrochloride (0.30 g, 916 μmol, 1.0 eq) was added to a round bottom flask charged with dichloromethane (3.00 mL) followed by addition of formaldehyde (371 mg, 4.58 mmol, 341 μL, 37.0% purity, 5.0 eq) and sodium triacetoxyborohydride (388 mg, 1.83 mmol, 2.0 eq) to the mixture. Stirred at 15° C. for 0.5 hr. The reaction was monitored by LCMS. Upon completion, the crude reaction was concentrated in vacuo. Took crude product to next step without purification. Obtained the title compound (3S,4R)-3-(2-(benzo[d][1,3]dioxol-5-yl)ethyl)-4-(4-fluorophenyl)-1-methylpiperidine (0.25 g, crude) as colorless oil. MS, ES⁺ m/z [M+H]⁺=342.2.

### Step 6: Preparation of (3S,4R)-3-(2-(benzo[d][1,3]dioxol-5-yl)ethyl)-4-(4-fluorophenyl)-1,1-dimethylpiperidin-1-ium iodide

(3S,4R)-3-(2-(benzo[d][1,3]dioxol-5-yl)ethyl)-4-(4-fluorophenyl)-1-methylpiperidine (0.05 g, 146 μmol, 1.0 eq) was added to a round bottom flask charged with acetone (0.35 mL). Iodomethane (41.5 mg, 292 μmol, 18.2 μL, 2.0 eq) was added dropwise to the mixture. Stirred at 20° C. for 3 hrs. The reaction was monitored by LCMS. Upon completion, the crude reaction was concentrated in vacuo. The crude product was purified by prep-HPLC (Phenomenex Gemini-NX 150×30-mm×5-μm, Condition: Water-MeOH) to give (3S,4R)-3-(2-(benzo[d][1,3]dioxol-5-yl)ethyl)-4-(4-fluorophenyl)-1,1-dimethylpiperidin-1-ium iodide (45 mg, 92.0 μmol, 62.8% yield, 98.9% purity) as yellow oil. MS, ES⁺ m/z [M+H]⁺=356.1. ¹H NMR (400 MHZ, DMSO-d6) δ 7.37-7.33 (m, 2H), 7.14 (t, J=8.8 Hz, 2H), 6.75 (d, J=8.0 Hz, 1H), 6.54 (d, J=2.0 Hz, 1H), 6.45 (dd, J=1.2 Hz, J=8.0 Hz, 1H), 5.93 (s, 2H), 3.73 (d, J=12.4 Hz, 1H), 3.52 (d, J=12.4 Hz, 1H), 3.44-3.38 (m, 1H), 3.25 (d, J=12.8 Hz, 1H), 3.21 (d, J=6.8 Hz, 1H), 2.56-5.52 (m, 1H), 2.47-2.42 (m, 1H), 2.21-2.10 (m, 1H), 1.83 (d, J=14.0 Hz, 1H), 1.31-1.17 (m, 2H).

### Example 18

### Assay Procedure

Uptake of serotonin (5-Hydroxytryptamine (5-HT)) in human HEK-293 cells embryonic kidney was measured according to the literature procedure by Gu H, Wall S. C. and Rudnick G. J Biol Chem. 1994 269, 7124. Using the following conditions:
Quantitation Method: Quantitation of [³H] Serotonin
Vehicle: 0.50% DMSO
Incubation Time/Temp: 15 minutes @ 25° C.
Incubation Buffer: 5 mM Tris —HCl, 7.5 mM HEPES, pH 7.1, 120 mM NaCl, 5.4 mM KCl,
1.2 mM CaCl₂, 1.2 mM MgSO₄, 5 mM DGlucose, 1 mM Ascorbic Acid

As provided in Table 1, representative compounds of formula (I) were found to be effective serotonin transporter SERT inhibitors at low concentrations. Any compound with minimum of 20% antagonism at 10 micromolar concentration as described above, is deemed a SERT inhibitor.

TABLE 1
Uptake of Serotonin (5-Hydroxytryptamine
(5-HT)) in human HEK-293 cells

| Example number | Activity Range^{†} |
|---|---|
| 1 | +++ |
| 2 | ++ |
| 3 | +++ |
| 4 | +++ |
| 5 | +++ |
| 6 | +++ |
| 7 | +++ |
| 8 | +++ |
| 9 | +++ |
| 10 | ++ |
| 11 | ++ |
| 12 | +++ |
| 13 | +++ |
| 14 | + |
| 15 | + |
| 16 | + |
| 17 | +++ |

^{†}In Table 1, a single plus (+) is associated with 20%-40% antagonism at 10 micromolar concentration. Two plus signs (++) is associated with 40%-50% antagonism at 10 micromolar concentration. Three plus signs (+++) is associated with greater than 50% antagonism at 10 micromolar concentration.

### Example 19

### Effects on Gastric Emptying in “IBS” and Control Mice

### 4.1 Animal Models and Rationale

In this experiment our aim was to study the effects of N-(2-((diaminomethylene)amino)ethyl)-4-(((3S,4R)-4-(4-fluorophenyl)piperidin-3-yl)methoxy)benzamide (example 8) in healthy mice as well as those with a model of gastrointestinal dysmotility (irritable bowel syndrome). The IBS model was induced by mild neonatal colorectal irritation in C₅₇B/6 mice; control mice received saline. Twenty μL of 0.5% acetic acid (AA) or saline were infused into the colorectum of mice during postnatal day 9-12. Pups were weaned at 3 weeks of age and allowed to grow up normally and tested after 8-12 weeks of age.

### 4.2 Treatment

Male IBS and control mice were treated with vehicle, Example 8 (10 mg/kg) and paroxetine (10 mg/kg) by gavage (10 ml/kg) once a day for 10 days. On the day 9, mice were fasted overnight. On the day 10, mice were treated with drugs followed by gavage with 0.3 ml of 0.05% phenol red in methylcellulose solution. Fifteen minutes later, mice were sacrificed and the stomach was removed after ligating the cardia and pylorus with hemostats. One mouse was sacrificed immediately after gavage of phenol red (0 time) used as control. The stomachs were homogenized and phenol red were extracted. After measuring the density of phenol red, the gastric empty was calculated as

$$Liquid G . E . ( \% ) = ( 1 - \frac{Absorbance of test sample}{Absorbance of baseline control ( G . E . at time 0 )} ) \times 100$$

### 4.3 Results

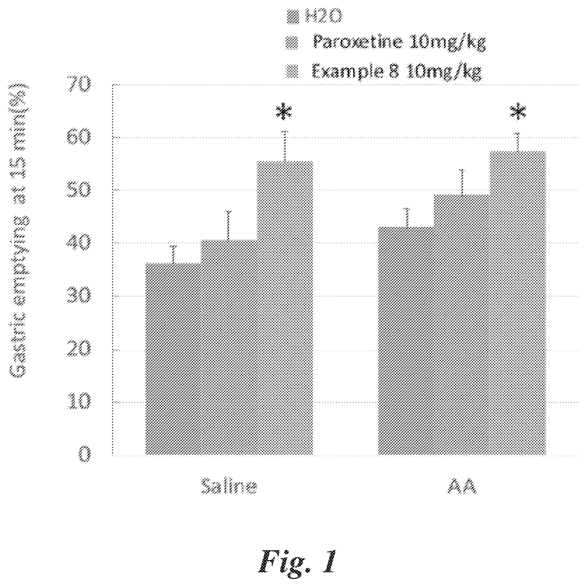

The data were analyzed by Two-WAY ANOVA followed by Student-Newman-Keuls post-hoc test. Two-WAY ANOVA showed that main effects of model F (1.32)=3.392, P=0.077; main effects of treatment F (2.32)=6.679, P=0.004; Interaction of model X treatment: F=0.658, P=0.526. Post-hoc test reveals that treatment with Example 8 significantly increased gastric emptying in both saline and IBS mice (P<0.05). This effect is not observed in the mice treated with paroxetine (FIG. 1), suggesting the effects of Example 8 are mediated by peripheral SSRIs. These results suggest that chronic treatment of example 8 can increase gastric emptying and may be effective as a treatment of gastroparesis.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Grider J R. Neurotransmitters mediating the intestinal peristaltic reflex in the mouse. J Pharmacol Exp Ther 2003; 307:460-7.
Mawe G M, Hoffman J M. Serotonin signalling in the gut—functions, dysfunctions and therapeutic targets. Nat Rev Gastroenterol Hepatol 2013; 10:473-86.
Wang S M, Han C, Bahk W M, et al. Addressing the Side Effects of Contemporary Antidepressant Drugs: A Comprehensive Review. Chonnam Med J 2018; 54:101-112.
Wei L, Singh R, Ha S E, et al. Serotonin Deficiency Is Associated With Delayed Gastric Emptying. Gastroenterology 2021; 160:2451-2466 e19.
Wang et al Neurogastroenterology and Motility 2012, 24, 560.
Rehavi and Gurwitz, WO 2007148341.
Coleman et al Nature 2016, 532, 334.
US2013197033 (A1) 2013 Aug. 1.
Segura, M. et al. Bioorg Chem. 2003, 31, 248.
Peshkova, M. et al. Electroanalysis, 2010, 22, 19, 2147.
Swanson, D. et al. Bioorg. Med. Chem. Lett, 2005, 16, 897.
WO0107036A1
WO2011070592A2
German, N. et al. Bioorg. Med. Chem. Lett. 2008, 18 1368.
WO2017040545 A1 2017 Mar. 9.
WO2018026866 A1 2018 Feb. 8.
WO2007072150A2
Aslam, S. N. et al. Tetrahedron 2006, 62, 4214.
Engevik M A, Luck B, Visuthranukul C, et al. Human-Derived Bifidobacterium dentium Modulates the Mammalian Serotonergic System and Gut-Brain Axis. Cellular and Molecular Gastroenterology and Hepatology 2021; 11:221-248.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (I): 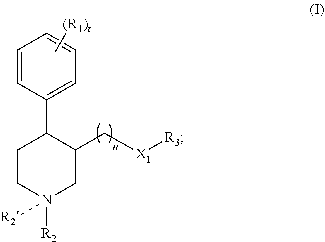
wherein:
( - - - ) indicates that the bond is present or absent;
n is an integer selected from 0, 1, 2, 3, and 4;
t is an integer selected from 0, 1, 2, and 3;
X₁ is oxygen or —CR₃R₄—, wherein R₃ and R₄ are each independently selected from H, substituted or unsubstituted straightchain or branched C₁-C₄ alkyl, halogen, substituted or unsubstituted aryl, alkoxyl, hydroxyl, carboxyl, nitro, amino, alkylamino, dialkylamino, sulfate, cyano, and mercapto;
each R₁ is independently selected from the group consisting of H, C₁-C₄ alkyl, C₁-C₂ alkoxyl, and halogen;
R₂ is H or substituted or unsubstituted straightchain or branched C₁-C₄ alkyl;
R₂′ is present or absent and, when present, is substituted or unsubstituted straightchain or branched C₁-C₄ alkyl, wherein, when present, the nitrogen atom to which it is bound has a positive charge;
R₃ is: 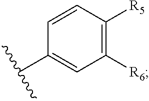
wherein:
R₅ and R₆ are each independently selected from the group consisting of H, —O—R₇, and —C(═O)—R₈, provided that at least one of R₅ or R₆ is not H, and wherein:
R₇ is selected from the group consisting of H, substituted or unsubstituted straightchain or branched C₁-C₄ alkyl, and —(CH₂)ₘ—R₉, wherein m is an integer selected from the group consisting of 0, 1, 2, 3, 4, 5, 6, 7, and 8;
R₉ is selected from the group consisting of —OR₁₀, —C(═O)—R₁₁, and —NR₁₂R₁₃, wherein:
R₁₀ is H or —(CH₂)ₚ—R₁₄, wherein p is an integer selected from the group consisting of 0, 1, 2, 3, 4, 5, 6, 7, and 8, R₁₄ is —OR₁₅ or —C(═O)—R₁₆, wherein R₁₅ and R₁₆ are each independently H or substituted or unsubstituted straightchain or branched C₁-C₄ alkyl;
R₁₁ is H or substituted or unsubstituted straightchain or branched C₁-C₄ alkyl;
R₁₂ and R₁₃ are each independently H or substituted or unsubstituted straightchain or branched C₁-C₄ alkyl;
R₈ is —OR 17 or —NR₁₈—(CH₂)_{q}—R₁₉, wherein:
q is an integer selected from 2, 3, 4, 5, 6, 7, and 8;
R₁₇ and R₁₈ are each independently H or substituted or unsubstituted straightchain or branched C₁-C₄ alkyl;
R₁₉ is —NR₂₀R₂₁ or —N═CR₂₂R₂₃, wherein:
R₂₀ and R₂₁ are each independently selected from the group consisting of H, substituted or unsubstituted straightchain or branched C₁-C₄ alkyl, and —C(═O)—NR₂₄R₂₅, wherein R₂₄ and R₂₅ are each independently H or substituted or unsubstituted straightchain or branched C₁-C₄ alkyl C₁-C₄ alkyl; and
R₂₂ and R₂₃ are each independently-NR₂₆R₂₇, wherein R₂₆ and R₂₇ are each independently H or substituted or unsubstituted straightchain or branched C₁-C₄ alkyl C₁-C₄ alkyl; or
wherein R₅ and R₆ together form a 5-membered heterocylic ring along with two carbons of the phenyl ring to which they are bound; and
pharmaceutically acceptable salts thereof.

2. The compound of claim 1, wherein the compound of formula (I) is:

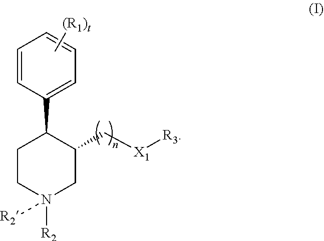

3. The compound of claim 1, wherein the compound of formula (I) is selected from the group consisting of:

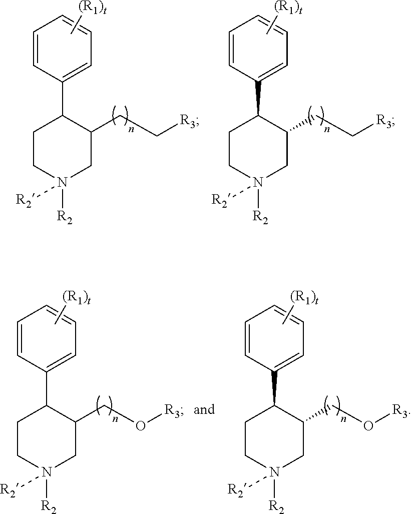

4. The compound of claim 1, wherein:
(a) R₅ is H and R₆ is —O—R₇ or —C(═O)—R₈; or
(b) R₆ is H and R₅ is —O—R₇ or —C(═O)—R₈.

5. The compound of claim 1, wherein:
n is 1;
X₁ is oxygen or —CR₃R₄—, wherein R₃ and R₄ are each H;
R₁ is halogen;
R₂ is H or substituted or unsubstituted straightchain or branched C₁-C₄ alkyl;
R₂′ is present or absent and, when present, is substituted or unsubstituted straightchain or branched C₁-C₄ alkyl, wherein, when present, the nitrogen atom to which it is bound has a positive charge;
R₃ is: 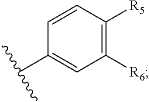
wherein:
R₆ is selected from the group consisting of H, —OH, and —C(═O)—OH;
R₅ is selected from the group consisting of H, —O—R₇, and —C(═O)—R₈;
R₇ is substituted or unsubstituted straightchain or branched C₁-C₄ alkyl or —(CH₂)ₘ—R₉, wherein m is an integer selected from the group consisting of 0, 1, 2, 3, 4, 5, 6, 7, and 8;
R₉ is selected from the group consisting of —OR₁₀, —C(═O)—R₁₁, and —NR₁₂R₁₃, wherein:
R₁₀ is —(CH₂)ₚ—R₁₄, wherein p is an integer selected from the group consisting of 0, 1, 2, 3, 4, 5, 6, 7, and 8, R₁₄ is —OR₁₅ or —C(═O)—R₁₆, wherein R₁₅ and R₁₆ are each H;
R₁₁ is H;
R₁₂ and R₁₃ are each independently H or substituted or unsubstituted straightchain or branched C₁-C₄ alkyl;
R₈ is —OR 17 or —NR₁₈—(CH₂)_{q}—R₁₉, wherein:
q is an integer selected from 2, 3, 4, 5, 6, 7, and 8;
R₁₇ and R₁₈ are each H;
R₁₉ is —NR₂₀R₂₁ or —N—CR₂₂R₂₃, wherein:
R₂₀ and R₂₁ are each independently selected from the group consisting of H, substituted or unsubstituted straightchain or branched C₁-C₄ alkyl, and —C(═O)—NR₂₄R₂₅, wherein R₂₄ and R₂₅ are each H; and
R₂₂ and R₂₃ are each independently-NR₂₆R₂₇, wherein R₂₆ and R₂₇ are each H; or
wherein R₅ and R₆ together form a 5-membered heterocylic ring along with two carbons of the phenyl ring to which they are bound; and
pharmaceutically acceptable salts thereof.

6. The compound of claim 1, wherein:
R₆ is H and R₅ is —C(═O)—R₈;
R₈ is —OR 17 or —NR₁₈—(CH₂)_{q}—R₁₉, wherein:
q is 2;
R₁₇ and R₁₈ are each H;
R₁₉ is —NR₂₀R₂₁ or —N═CR₂₂R₂₃, wherein:
R₂₀ and R₂₁ are each independently selected from the group consisting of H, substituted or unsubstituted straightchain or branched C₁-C₄ alkyl, and —C(═O)—NR₂₄R₂₅, wherein R₂₄ and R₂₅ are each H; and
R₂₂ and R₂₃ are each independently-NR₂₆R₂₇, wherein R₂₆ and R₂₇ are each H.

7. The compound of claim 1, wherein R₆ is H and R₅ is —O—R₇, wherein:
R₇ is —(CH₂)ₘ—R₉, wherein m is 1, 2, or 3;
R₉ is selected from the group consisting of —OR₁₀, —C(═O)—R₁₁, and —NR₁₂R₁₃, wherein:
R₁₀ is —(CH₂)ₚ—R₁₄, wherein p is 1 or 2; R₁₄ is —OR 15 or —C(═O)—R₁₆, wherein R₁₅ and R₁₆ are each H;
R₁₁ is H; and
R₁₂ and R₁₃ are each independently H or substituted or unsubstituted straightchain or branched C₁-C₄ alkyl.

8. The compound of claim 1, wherein R₅ and R₆ together form a 1,3-dioxolane ring with two carbons of the phenyl group to which they are attached.

9. The compound of claim 1, wherein the compound of formula (I) is selected from the group consisting of:
(3S,4R)-3-(2-(benzo[d][1,3]dioxol-5-yl)ethyl)-4-(4-fluorophenyl)-1,1-dimethylpiperidin-1-ium iodide;
5-(((3S,4R)-4-(4-fluorophenyl)piperidin-3-yl)methoxy)-2-methoxybenzoic acid;
4-(((3S,4R)-4-(4-fluorophenyl)piperidin-3-yl)methoxy)-2-hydroxybenzoic acid;
3-(((3S,4R)-4-(4-fluorophenyl)piperidin-3-yl)methoxy)benzoic acid;
4-(((3S,4R)-4-(4-fluorophenyl)piperidin-3-yl)methoxy)benzoic acid;
N-(2-((diaminomethylene)amino)ethyl)-4-(((3S,4R)-4-(4-fluorophenyl)piperidin-3-yl)methoxy)benzamide;
4-(((3S,4R)-4-(4-fluorophenyl)piperidin-3-yl)methoxy)-N-(2-ureidoethyl)benzamide;
N-(2-aminoethyl)-4-(((3S,4R)-4-(4-fluorophenyl)piperidin-3-yl)methoxy)benzamide;
N-(2-(dimethylamino)ethyl)-4-(((3S,4R)-4-(4-fluorophenyl)piperidin-3-yl)methoxy)benzamide;
N-(2-(dimethylamino)ethyl)-4-(((3S,4R)-4-(4-fluorophenyl)piperidin-3-yl)methoxy)-N-methylbenzamide;
3-(4-(((3S,4R)-4-(4-fluorophenyl)piperidin-3-yl)methoxy)phenoxy)-N,N-dimethylpropan-1-amine;
2-(2-(4-(((3S,4R)-4-(4-fluorophenyl)piperidin-3-yl)methoxy)phenoxy)ethoxy)ethan-1-ol;
2-(2-(4-(((3S,4R)-4-(4-fluorophenyl)piperidin-3-yl)methoxy)phenoxy)ethoxy)acetic acid;
2-(4-(((3S,4R)-4-(4-fluorophenyl)piperidin-3-yl)methoxy)phenoxy)ethan-1-amine;
2-(4-(((3S,4R)-4-(4-fluorophenyl)piperidin-3-yl)methoxy)phenoxy)-N,N-dimethylethan-1-amine;
2-(4-(((3S,4R)-4-(4-fluorophenyl)piperidin-3-yl)methoxy)phenoxy)ethan-1-ol; and
2-(4-(((3S,4R)-4-(4-fluorophenyl)piperidin-3-yl)methoxy)phenoxy)acetic acid.

10. The compound of claim 1, wherein the compound of formula (I) comprises a pharmaceutically acceptable salt selected from the group consisting of:
4-(((3S,4R)-4-(4-fluorophenyl)piperidin-3-yl)methoxy)benzoic acid hydrochloride;
3-(((3S,4R)-4-(4-fluorophenyl)piperidin-3-yl)methoxy)benzoic acid hydrochloride;
4-(((3S,4R)-4-(4-fluorophenyl)piperidin-3-yl)methoxy)-2-hydroxybenzoic acid hydrochloride (LI-987);
5-(((3S,4R)-4-(4-fluorophenyl)piperidin-3-yl)methoxy)-2-methoxybenzoic acid hydrochloride;
N-(2-aminoethyl)-4-(((3S,4R)-4-(4-fluorophenyl)piperidin-3-yl)-methoxy)benzamide hydrochloride;
N-(2-(dimethylamino)ethyl)-4-(((3S,4R)-4-(4-fluorophenyl)piperidin-3-yl)methoxy)benzamide hydrochloride;
N-(2-(dimethylamino)ethyl)-4-(((3S,4R)-4-(4-fluorophenyl)piperidin-3-yl)methoxy)-N-methylbenzamide hydrochloride
N-(2-((diaminomethylene)amino)ethyl)-4-(((3S,4R)-4-(4-fluorophenyl)piperidin-3-yl)methoxy)benzamide;
4-(((3S,4R)-4-(4-fluorophenyl)piperidin-3-yl)methoxy)-N-(2-ureidoethyl)benzamide hydrochloride;
2-(4-(((3S,4R)-4-(4-fluorophenyl)piperidin-3-yl)methoxy)phenoxy)-ethan-1-amine hydrochloride;
2-(4-(((3S,4R)-4-(4-fluorophenyl)piperidin-3-yl)methoxy)phenoxy)-N,N-dimethylethan-1-amine hydrochloride;
3-(4-(((3S,4R)-4-(4-fluorophenyl)piperidin-3-yl)methoxy)phenoxy)-N,N-dimethylpropan-1-amine hydrochloride;
2-(4-(((3S,4R)-4-(4-fluorophenyl)piperidin-3-yl)methoxy)phenoxy)ethan-1-ol hydrochloride;
2-(2-(4-(((3S,4R)-4-(4-fluorophenyl)piperidin-3-yl)methoxy)phenoxy)ethoxy)ethan-1-ol hydrochloride;
2-(4-(((3S,4R)-4-(4-fluorophenyl)piperidin-3-yl)methoxy)phenoxy)acetic acid hydrochloride;
2-(2-(4-(((3S,4R)-4-(4-fluorophenyl)piperidin-3-yl)methoxy)phenoxy)-ethoxy)acetic acid hydrochloride; and
(3S,4R)-3-(2-(benzo[d][1,3]dioxol-5-yl)ethyl)-4-(4-fluorophenyl)-1,1-dimethylpiperidin-1-ium iodide.

11. A pharmaceutical formulation comprising a compound of claim 1.

12. A method for treating a disease, condition, or disorder associated with serotonin (5-HT) signaling, the method comprising administering a therapeutically effective compound of claim 1 to a subject in need of treatment thereof.

13. The method of claim 12, comprising inhibiting serotonin/5-HT transporter (SERT).

14. The method of claim 13, wherein inhibiting SERT increases an extracellular concentration of 5-HT.

15. The method of claim 12, wherein the disease, condition, or disorder associated with 5-HT signaling comprises a gastroenterological disorder.

16. The method of claim 15, wherein the gastroenterological disorder is selected from colitis, irritable bowel syndrome (IBS), constipation, diarrhea, and gastroparesis.

17. The method of claim 12, wherein the disease, condition, or disorder associated with 5-HT signaling comprises an extra-gastrointestinal disorder.

18. The method of claim 17, wherein the extra-gastrointestinal disorder is selected from asthma, migraine, itching, osteoporosis, anxiety, depression and impaired cognition.
